(19) United States
(12) Patent Application Publication
(10) Pub. No.: US 2024/0390037 A1
(43) Pub. Date: Nov. 28, 2024

(54) EXTERNAL FIXATION DEVICE AND/OR METHOD FOR A FRACTURED LIMB

(71) Applicant: Nikolaj WOLFSON, San Francisco, CA (US)

(72) Inventor: Nikolaj WOLFSON, San Francisco, CA (US)

(21) Appl. No.: 18/671,797

(22) Filed: May 22, 2024

Related U.S. Application Data
(60) Provisional application No. 63/468,030, filed on May 22, 2023.

(51) Int. Cl. A61B 17/64; A61B 17/00; A61B 17/66; A61B 90/00

(52) U.S. Cl. CPC A61B 17/6416; A61B 17/66; A61B 2017/00991; A61B 2090/064

(57) ABSTRACT

Embodiments of the present disclosure relate generally to systems and methods for stabilizing a fracture in a long bone, and more particularly, a fixation device configured to stabilize a fracture in a long bone. The fixation device includes a frame a frame including an elongate member, a first carrier configured to be coupled to a first fixator member and configured to be coupled to the elongate member, and a second carrier configured to be coupled to a second fixator member and configured to be coupled to the elongate member.

### CROSS-REFERENCE TO RELATED APPLICATIONS

This application claims the benefit of U.S. Provisional Patent Application No. 63/468,030, filed May 22, 2023, and hereby incorporates by reference herein the entire contents of this application.

### TECHNICAL FIELD

The present disclosure, in some embodiments thereof, relates to an orthopedic fixation device and/or method of treatment for treating a damaged limb, for example a fractured limb, a limb with wounds and/or burns and, more particularly, but not exclusively, to a fixation device for immediate, on-site and/or delayed use which can be selectively adjusted, assembled and/or deployed as per the need.

### BACKGROUND

Over 50% of injuries in mass casualties, war, and other man-made and natural disasters involve extremities of a patient's body. External fixation is one way these injuries and other severe limb fractures typically are treated, for example, by using unilateral, hybrid, and circular external fixators. Complex fractures are typically treated with hybrid and/or circular external fixators. However, such external fixators, as well as other standard surgical-setting equipment, such as fracture tables, are very complex, expensive, require high level of expertise and often not available for rapid stabilization of fractures in certain field locations, such as, for example, as a result of mass casualties, war, and other man-made and natural disasters.

Severe fractures of a patient's extremities, especially of the lower limbs, can be accompanied by damage to the surrounding soft tissue. Such soft tissue damage may be due to open fractures, open fractures with burns, gunshot wounds, and so forth. In embodiments in which the soft tissue near the fracture is badly damaged, it is advantageous to minimize further damage to the soft tissue when treating the fracture. Therefore, surgical methods typically used to treat fractures, for example, intramedullary screws and/or intramedullary plates, are typically not used to treat fractures with surround soft tissue damage. Instead, external fixation systems are used to support the bone fragments of the fractured bone while minimizing further damage to the soft tissue.

### SUMMARY

In some embodiments, a fixation device configured to stabilize a fracture in a long bone includes a frame, a first carrier, and a second carrier. The frame includes an elongate member. The first carrier is configured to be coupled to a first fixator member and configured to be coupled to the elongate member. The second carrier is configured to be coupled to a second fixator member and configured to be coupled to the elongate member.

In some embodiments, a method of treating a facture on a long bone having a first end and a second end includes positioning a first carrier along an elongate member of a frame of a fixation device such that the first carrier is oriented to be proximate the first end of the long bone when the frame is coupled is coupled to the long bone; positioning a second carrier along the elongate member of the frame such that the second carrier is oriented to be proximate the second end of the long bone when the frame is coupled is coupled to the long bone; coupling a first fixator member to the first carrier; coupling a second fixator member to the second carrier; and coupling the first fixator member and the second fixator member to the fracture.

In some embodiments, a portable fixation table configured to stabilize a fracture in a long bone includes a frame, a first carrier, and a second carrier. The frame includes an elongate member. The first carrier is configured to be coupled to a first fixator member and is configured to be coupled to the elongate member. The second carrier configured to be coupled to a second fixator member and is configured to be coupled to the elongate member. The frame is configured to be moveable between a collapsed state in which the first and second fixator members lie in a plane that is generally parallel to a plane in which the elongate member lies, and a deployed state in which the first fixator member lies in a plane that is generally perpendicular to the plane in which the elongate member lie and in which the second fixator member lies in a plane that is generally perpendicular to the plane in which the elongate member lies.

### BRIEF DESCRIPTION OF THE DRAWINGS

Some embodiments of the disclosure are herein described, by way of example only, with reference to the accompanying drawings. With specific reference now to the drawings in detail, it is stressed that the particulars shown are by way of example and for purposes of illustrative discussion of embodiments of the disclosure. In this regard, the description taken with the drawings makes apparent to those skilled in the art how embodiments of the disclosure may be practiced.

FIG. 1 illustrates an example fixation device in accordance with various features of the present disclosure.

FIG. 2 illustrates a front view of the fixation device of FIG. 1 in accordance with various features of the present disclosure.

FIG. 3 illustrates a perspective view of an example frame of the fixation device of FIG. 1 in accordance with various features of the present disclosure.

FIG. 4 illustrates another example fixation device in accordance with various features of the present disclosure.

FIG. 5 illustrates a perspective view of the example fixation device of FIG. 4 in an unfolded configuration in accordance with various features of the present disclosure.

FIG. 6 illustrates another perspective view of the example fixation device of FIG. 4 in an unfolded configuration in accordance with various features of the present disclosure.

FIG. 7 illustrates a detail view of an example fixator member coupled to the fixation device of FIG. 4 in accordance with various features of the present disclosure.

FIG. 8 is a detail view of a wire and a wire holder coupled to the fixator member of FIG. 7 in accordance with various features of the present disclosure.

FIG. 9 illustrates a front view of another example fixator member and carrier for use with the fixation device of FIG. 4 in accordance with various features of the present disclosure.

FIG. 10 illustrates a perspective view of the example fixator member and carrier of FIG. 9 in accordance with various features of the present disclosure.

FIG. 11 illustrates a side view of the example fixator member and carrier of FIG. 9 in accordance with various features of the present disclosure.

FIG. 12 illustrates a perspective view of an example carrier for use with the fixation device of FIG. 4 in accordance with embodiments of the present disclosure.

FIG. 13 illustrates a side view of carrier of FIG. 12 in accordance with embodiments of the present disclosure.

FIG. 14 illustrates a perspective view of another example carrier for use with the fixation device of FIG. 4 in accordance with various features of the present disclosure.

FIG. 15 illustrates a perspective view of yet another carrier coupled to a fixator member for use with the fixation device of FIG. 4 in accordance with various features of the present disclosure.

FIG. 16 illustrates an example clamp for use with the fixation device of FIG. 4 in accordance with various features of the present disclosure.

FIG. 17 illustrates an example a flowchart of a method 2000 for treating a limb fracture using a fixation device, such as the fixation devices of FIG. 1, FIG. 4, or FIG. 35 in accordance with various features of the present disclosure.

FIG. 18 illustrates an example fracture that can be treated with the fixation device of FIG. 1, FIG. 4, or FIG. 35 in accordance with various features of the present disclosure.

FIG. 19 illustrates another example fracture that can be treated with the fixation device of FIG. 1, FIG. 4, or FIG. 35 in accordance with various features of the present disclosure.

FIG. 20 illustrates a schematic representation of a limb coupled to a fixator member of the fixation device of FIG. 4 in accordance with various features of the present disclosure.

FIG. 21 illustrates a schematic representation of a limb coupled to the fixation device of FIG. 4 in accordance with various features of the present disclosure.

FIG. 22 illustrates a fixator member including a rotary portion in accordance with various features of the present disclosure.

FIG. 23 illustrates a perspective view of the fixator member of FIG. 23 in accordance with various features of the present disclosure.

FIG. 24 illustrates another fixator member including a rotary portion in accordance with various features of the present disclosure.

FIG. 25 illustrates another fixator member in accordance with various features of the present disclosure.

FIG. 26 illustrates an example clamp configured to couple a fixator member to a frame of a fixation device in accordance with various features of the present disclosure.

FIG. 27 illustrates a first configuration the clamp of FIG. 27 coupled to an example fixator member in accordance with various features of the present disclosure.

FIG. 28 illustrates a second configuration of the clamp of FIG. 31 coupled to an example fixator member in accordance with various features of the present disclosure.

FIG. 29 illustrates a flowchart of a method of using a fixation device such for immediate damage control and for long term treatment in accordance with various features of the present disclosure.

FIG. 30 illustrates a perspective view of a fixation device coupled to a limb and including a frame and a hexapod external fixator in accordance with various features of the present disclosure.

FIG. 31 illustrates a perspective view of the fixation device of FIG. 30 in accordance with various features of the present disclosure.

FIG. 32 illustrates a perspective view of the hexapod external fixator of FIG. 33 in accordance with various features of the present disclosure.

FIG. 33 illustrates a schematic diagram of an example kit for immediate treatment of a fractured limb in accordance with various features of the present disclosure.

FIG. 34 illustrates a fixation device having an offset in accordance with various features of the present disclosure.

FIG. 35 illustrates a perspective view of an example fixation device in accordance with various features of the present disclosure.

FIG. 36 illustrates a top view of the fixation device of FIG. 35 in accordance with various features of the present disclosure.

FIG. 37 illustrates a perspective view of the fixation device of FIG. 35 including a frame coupled to fixator members in accordance with various features of the present disclosure.

FIG. 38 illustrates a perspective view of the frame of the fixator device of FIG. 35 coupled to fixator members and coupled to a limb of a patient in accordance with various features of the present disclosure.

FIG. 39 illustrates another perspective view of the frame of the fixator device of FIG. 35 coupled to fixator members and coupled to a limb of a patient in accordance with various features of the present disclosure.

FIG. 40 illustrates a section view of FIG. 39 taken along lines 40-40 in accordance with various features of the present disclosure.

FIG. 41 illustrates a front view of a fixator member for use with a fixation device in accordance with various features of the present disclosure.

FIG. 42 illustrates a front view the fixator member of FIG. 41 coupled to a closure element in accordance with various features of the present disclosure.

FIG. 43 illustrates a front view of the fixator member and closure elements of FIG. 41 coupled to wires in accordance with various features of the present disclosure.

FIG. 44 illustrates a side view of FIG. 43 in accordance with various features of the present disclosure.

FIG. 45 illustrates a bottom view of FIG. 43 in accordance with various features of the present disclosure.

FIG. 46 illustrates a schematic representation of the frame of FIG. 35 in accordance with various features of the present disclosure.

FIG. 47 shows an example system diagram of various hardware components and other features for use with the fixation device of FIG. 35, according to embodiments of the present disclosure.

FIG. 48 shows a representative block diagram of various example system components for use with the fixation device of FIG. 35, according to embodiments of the present disclosure.

FIG. 49 shows a perspective view of the fixation device of FIG. 35 including carrier members coupled to stabilizing pads, according to embodiments of the present disclosure.

FIG. 50 shows a front view of a stabilizing pad of FIG. 49, according to embodiments of the present disclosure.

FIG. 51 shows a perspective view of the stabilizing pad of FIG. 49, according to embodiments of the present disclosure.

FIG. 52 shows a front perspective view of a carrier for use with the fixation device of FIG. 35, according to embodiments of the present disclosure.

FIG. 53 shows a rear view of the carrier for use with the fixation device of FIG. 35, according to embodiments of the present disclosure.

FIG. 54 shows a fixation device coupled between a patient's ankle and the patient's pelvis according to embodiments of the present disclosure.

### DESCRIPTION OF SPECIFIC EMBODIMENTS OF THE INVENTION

The basic element is a portable platform that will provide a basis to any type of external fixation. This platform consists of a number of telescoping, or solid tubes made out of any material, be it metal or composite, of any geometrical cross-section (round, square, triangular or other) which are bridged by a carriage, or any other attached elements forming stable platform/foundation. In addition to providing an apparatus suitable for external fixation, the present invention also provides a portable, field-ready substitute for certain conventional surgical-setting equipment, such as, for example, a fracture table.

In some embodiments, and as will be described in greater detail below with specific reference to the Figures and to the features shown therein, a fixation device configured to treat fractures in long bones includes a frame including two or more elongate members. At least a proximal carrier and a distal carrier are movably coupled between the elongate members. The proximal and distal carrier are each configured to secure a fixator member therebetween. Optionally, additional carriers (configured to secure additional fixator members) can be coupled between the proximal carrier and the distal carrier. The frame can act as a stable platform/portable operating table, or fracture table, with which a healthcare practitioner can stabilize, reduce and fix fractures in locations where access to such equipment typically is limited. For example, the practitioner can adjust a length of the frame based on a length of the limb including the fracture, position the carriers to best support the fractured limb, and couple the fixator members to the carriers. At this point, the fractured limb can be positioned within the fixation device. The frame, carriers, and fixator members can secure and support the fractured limb as wires are inserted into the limb to couple the fixator members to the limb, fractured bone fragments are manipulated to be re-aligned, and/or pins are used to reposition fractured bone fragments such that a single practitioner can stabilize a fractured limb without assistance. In some embodiments, stabilizing pads may be coupled to the fixator members and/or the carriers, which can provide further stabilization to the limb as the fixation device is being coupled to the patient and/or as the bone fragments are being manipulated by the practitioner.

As used herein, the phrases “healthcare practitioner” and “practitioner” are intended to encompass any type of healthcare practitioner treating the patient with the fixation devices described herein, including doctors, nurses, medical assistants, surgical assistants, emergency responders, caregivers, and so forth.

It is to be understood that the disclosure is not necessarily limited in its application to the details of construction and the arrangement of the components and/or methods set forth in the following description and/or illustrated in the drawings. The disclosure is capable of other embodiments or of being practiced or carried out in various ways.

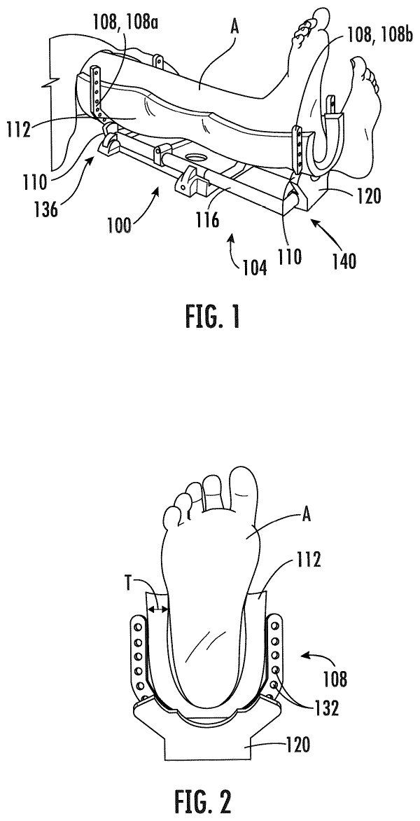

FIGS. 1-2 schematically illustrate a fractured leg A received within a fixation device 100, according to some embodiments.

In some embodiments, fixation device 100 includes a frame 104, one or more fixator members or bows 108, and a stabilizing pad 112. In some embodiments, the frame 104 may interchangeably be referred to as a portable fixation table.

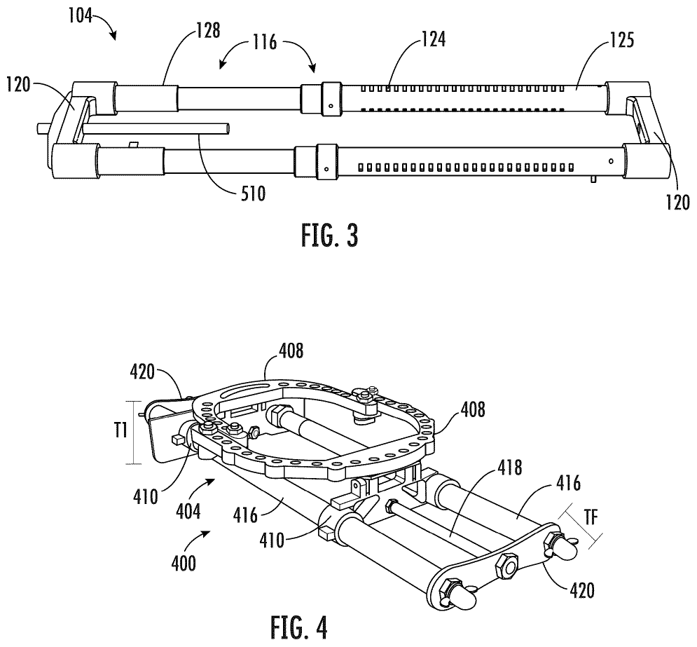

The frame 104 is configured to receive and hold at least a portion of a limb of a patient. In some embodiments, the limb includes a leg portion from the knee down. As shown in FIG. 3, in some embodiments, the frame 104 comprises a plurality of elongate members 116 (such as rods, shafts, tubes, and so forth) that are coupled together at their proximal and distal ends by a transverse connector 120. The transverse connector 120 may include a plate, rod, shaft, or other connector rigid connector configured to extend between the elongate members 116. In the configuration illustrated in FIG. 3, the two elongate members 116 are aligned parallel to each other such that the frame 104 has a substantially open rectangular configuration.

With continued reference to FIG. 3, in some embodiments, the elongate members 116 have a telescoping tube-in-tube configuration such that a length of the frame 104 can be adjusted based on a length of the limb being treated. In such embodiments, the elongate members 116 include locking features 124 configured to maintain the elongate members 116 at a particular length. In the embodiment illustrated in FIG. 3, the locking features 124 include holes in an outer element 125 of the elongate members 116 configured to receive a detent mechanism of an inner tube. In other configurations, the locking features 124 may include other elastic mechanical interference mechanisms, or locking mechanism, such as a locking screw or a detent pin. In some embodiments, the frame 104 can be adjusted such that a length of the frame 104 is substantially similar to a length of the fractured limb. For example, for a fracture in the tibia and/or fibula, the frame 104 can be adjusted such that the length of the frame 104 is substantially similar to a length of the patient's tibia and/or fibula. In some embodiments, a length of the frame 104 can extend from the patient's ankle to the patient's pelvis (FIG. 54).

In some embodiments, a fixed-length or adjustable-length extension 128 may be coupled to an end of the elongate members 116 to further lengthen the frame 104. In such embodiments, the extension 128 may be selectively and removably coupled to the elongate members 116. The extension 128 may be coupled to the elongate members 116 when preparing the frame 104 for deployment, for example if the frame 104 is not long enough to extend along a length of the limb portion to be treated.

Optionally, in embodiments in which the length of the frame 104 is adjustable, the elongate members 116 may include markings to assist with sizing the length of the frame 104 based on the size of the fractured limb.

In some embodiments of the disclosure, the transverse connector 120 may include a knob (not shown) configured to adjust the length of elongate members 116. For example, a screw (not shown) may be located inside the elongate members 116 and rotation of the screw (relative to a threading, such as a nut) via the knob, causes lengthening or shortening of the elongate members 116, thereby increasing or decreasing a length of the frame 104.

Returning to FIGS. 1-2, the one or more fixator members 108 are coupled to the frame 104. In the illustrated embodiments, the fixator member 108 is shown as an open circle. In other embodiments, other shapes such as arcs, U-shaped elements, annular rings, half U-shapes, horseshoe shapes, half-horseshoe shapes, L-shapes, reverse omega shapes, and so forth may be used.

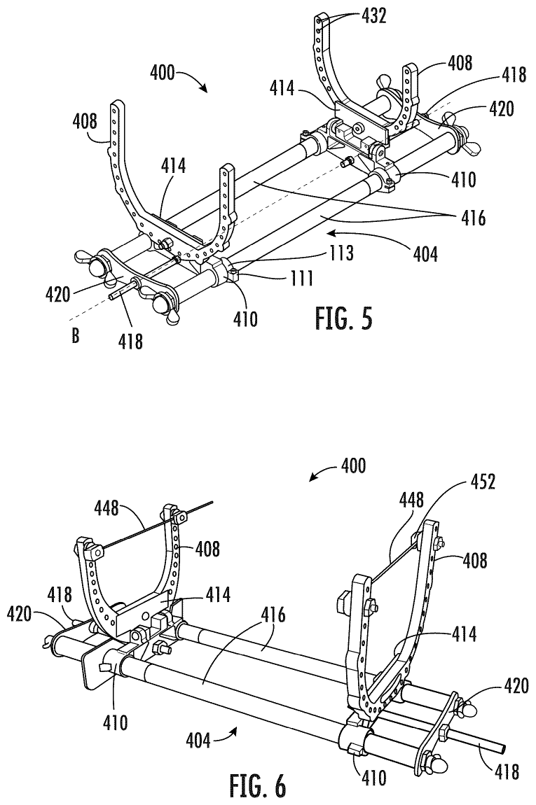

In some embodiments, the fixator member(s) include a plurality of holes 132 in which a holder 452 (FIG. 6) may be placed at selected holes for holding either a wire 448 (FIG. 6) or a half-pin (not shown), according to some embodiments, for guiding wires and/or pins through. As is described in greater detail below, these wires and/or pins cooperate with the fixator members 108 and the frame 104 to support bone fragments in the limb. In some embodiments, the fixator members 108 may be coupled to the frame 104 by carriers 110, for example, by clamping devices, threaded connectors such as screws, detent pins, and so forth. In some embodiments, the fixator members 108 and the carriers 110 are integrally formed to a one-piece construction. In some embodiments, the carriers 110 may be fixedly coupled to the frame 104. In some embodiments, the carriers 110 may be configured to slide along the frame 104, and to be firmly secured to the frame 104 at a selected axial position (for example by fastening of fastener 111 (FIG. 5) such as a pin, screw or clip). Optionally, fastener 111 tightens the carrier portions 113 (FIG. 5) which are seated on the frame 104, to lock the carrier portions 113 into place. In some embodiments, the elongate members 116 may include markings to assist with appropriate positioning of the carrier 110, relative to the frame 104 and the limb A, based on the fracture location, the ends of the injured limb, and so forth.

In some embodiments, the fixator members 108 are removably coupled to the frame 104. In other embodiments, the fixator members 108 can be pre-fixed (optionally, integrally attached) to the frame. As shown in FIG. 1, typically, a first or proximal fixator member 108a is coupled at or proximate a proximal end 136 of the frame 104 and a second or distal fixator member 108b is coupled at or proximate a distal end 140 of the frame 104 such that the fractured portion of the limb is positioned between the first and second fixator members 108a, 108b. In some embodiments, the proximal fixator member 108a extends from a most proximal horizontal edge of the frame 104, and the distal fixator member extends from a most distal horizontal edge of the frame 104. In some embodiments, additional fixator members 108 may be positioned between the proximal fixator member 108a and the distal fixator member 108b.

As shown in FIGS. 1-2, in some embodiments, the fixation device 100 includes a limb support, for example the stabilizing pad 112. In some embodiments, the stabilizing pad 112 is shaped and configured to provide a counterforce to supports the limb A positioned within the fixation device 100, potentially reducing or preventing movement of the limb A relative to the frame 104 and/or fixator members 108.

In some embodiments, the stabilizing pad 112 extends along the limb A portion such that the stabilizing pad 112 prevents contact between the limb tissue and fixation device 100 and/or distributes the pressure forces acting between the device frame 104 and the limb portion A. In some embodiments, the stabilizing pad 112 is formed of a continuous surface along which the forces may be more homogenously distributed.

In some embodiments, the stabilizing pad 112 extends along the length of the frame 104, to receive the fractured limb portion therein. In some embodiments, the stabilizing pad 112 may be secured to one or more fixator members 108, carriers 110, and/or a portion of the frame 104 via a clamp. In some embodiments, the stabilizing pad 112 is constructed from, or otherwise includes, a foam material, a resin material, and/or other material or structure, such as a water or air containing cushion. In some embodiments, in use, the practitioner positions the fractured limb portion such that it is rested against the stabilizing pad 112. The stabilizing pad 112 is configured position the soft tissue of the patient's limb, relative to the fixator member 108 and/or the frame 104, by a spacing S. In some aspects, the spacing S is from about 5 millimeters (mm) to 100 mm. The stabilizing pad 112 is stiff and/or elastic enough to maintain the spacing S between the soft tissue of the patient's limb and the fixator member 108 and/or the frame 104 even under the weight of the patient's limb, if resting thereon.

In some embodiments, the stabilizing pad 112 surrounds at least 30%, at least 40%, at least 50%, at least 70% or intermediate, larger or smaller percentage of a perimeter of the limb portion (e.g. a perimeter of the lower leg), at least partially encompassing the limb. In some embodiments, the stabilizing pad 112 is folded against an inner surface 144 of the fixator members 108 extending from the frame, forming an inner lining along the device 100. The stabilizing pad 112 may have a length of, for example, between 5 and 100 cm, for example, between 10 and 60 cm. Optionally, the stabilizing pad 112 is sized to match a limb length. Optionally, the stabilizing pad 112 is scored or otherwise weakened so it can be torn or cut to length and/or have windows formed therein to only contact part of the limb. Optionally, the stabilizing pad 112 is stiff enough to support a limb between adjacent fixator members 108. Optionally or additionally, separate stabilizing pads 112 are coupled to for each fixator member. Optionally or alternatively, the stabilizing pad 112 includes one or more loops or hooks to hang the stabilizing pad 112 from a fixator member 108 and/or to tension the stabilizing pad 112 between adjacent fixator members 108.

A potential advantage of using the stabilizing pad 112 is that it at least partially encompasses the limb portion A, thereby stabilizing the limb portion A relative to the device frame 104 and facilitating the fixation process. A further advantage of using the stabilizing pad 112 is that it may allow, for example, for a single practitioner to perform the fixation without requiring additional personnel or other external means for stabilizing the limb A. In such configurations, the stabilizing pad 112 and the frame 104 may form a stationary “hub” which supports the limb A as the limb A is immobilized, facilitating the introduction of wires and/or pins through the bone. In this aspect of the invention, the device 100 provides the stabilizing effect of a fracture table, but is light-weight, collapsible and portable, thereby providing the stabilizing effect of a fracture table in settings, such emergency settings or a battlefield, where such equipment is typically not available.

Another potential advantage of a stabilizing pad 112 may include reducing or avoiding damage to soft tissue surrounding the bone, as the limb portion A rests against the stabilizing pad 112 and is at least partially immobilized by the stabilizing pad 112.

In some embodiments, the stabilizing pad 112 may be formed of, or include, for example, foam, gel, soft plastic, and/or other materials. For example, a surface conforming pad may be made using a bean bag (e.g., filled with small balls or other smooth shapes). In another example, the stabilizing pad may be made from or include an inflatable component. Optionally, the stiffness of the stabilizing pad 112 can be modified by modifying an inflation amount and/or pressure. Optionally or alternatively, the stabilizing pad 112 is formed or has an inner surface of memory foam. In some embodiments, the stabilizing pad 112 comprises a contoured surface shaped to fit the limb. In some embodiments, the stabilizing pad 112 defines an upper surface that is configured to be compressed down by the limb A for example so as to form a lining between the limb A and the frame 104 and/or the fixator members 108 which conforms to the specific shape of the limb A.

In some embodiments of the disclosure, the stabilizing pad 112 is made of or includes a water soluble element. This water soluble element may assist in removing the stabilizing pad 112.

In some embodiments, a thickness T of the stabilizing pad 112 is between 1.5 cm-3 cm, 1 cm-2 cm, 0.5 cm-6 cm or intermediate, higher or lower thickness. In some embodiments, the thickness T of the stabilizing pad 112 is selected so that upon removal of the stabilizing pad 112, there will remain a sufficient space between the limb A and the device frame 104 and/or the fixator members 108 to accommodate potential swelling of the limb A.

FIGS. 4-6 show a fixation device 400 according to an embodiment of the present disclosure. Like parts between the fixation device 100 and the fixation device 400 will be shown using like numbering. The fixation device 400 is only described in detail herein to the extent that it differs from the fixation device 100. FIG. 4 shows the fixation device 400 in a collapsed, folded configuration and FIGS. 5-6 show the fixation device 400 in an unfolded, deployable configuration.

In the configuration shown in FIGS. 4-6, the carrier 410 includes a holder such as a coupling plate 414 that can be rotated relative to the carrier 410. The fixator member 408 is coupled to the carrier 410 via the coupling plate 414, such that the fixator member 408 can be rotated about an axis A that is transverse to a longitudinal axis B of the frame 404.

In some embodiments, the fixation device 400 is configured to be unfolded from a collapsed state, such as shown in FIG. 4, to an open, deployable state, such as shown in FIGS. 5-6. In some embodiments, in the folded state, one or more fixator members 408 are folded onto the frame 404 such that the fixator members 408 are substantially parallel to the frame. In some embodiments, in the folded state, a thickness T1 of fixation device (such including the frame and fixator members folded on top of it) does not exceed, for example, 4 cm, 6 cm, 10 cm or intermediate, higher or smaller thickness. In some embodiments, the frame 404 has a thickness T_{F} of between 2-6 cm. In some embodiments, a thickness TI of the frame 404 coupled to fixator members 408 in a folded configuration is about 5-20 and/or 3-10 cm. In some embodiments, the folded fixation device 400 can be easily stored and carried around, occupying a relatively small volume as compared to its open state. While in some embodiments the frame 404 and/or fixator members 408 are formed of stainless steel (e.g., which can go through an autoclave), other materials may be used, for example, plastic, carbon fiber and composite materials. This may reduce weight and/or aid in disposability.

In some embodiments, in the open state, the fixator members 408 can extend along a plane crossing the plane defined by the frame 404, for example, to be vertical to the frame 404. In some embodiments, each fixator member 408 is coupled to the frame 404 by the carrier 410. In some embodiments, the carrier 410 is moveable (e.g. slidable) along the elongate members 416 of the frame 404, so that the fixator member 408 is selectively positioned along the length of the frame 404.

In some embodiments, movement of the carrier 410 (along with the fixator member 408 attached thereto) is carried out by pulling and/or pushing on one end of a longitudinal adjuster 418 such as a rod, pin or screw which is coupled to the carrier 410. In some embodiments, the longitudinal adjuster 418 extends through a designated hole in the frame 404 (e.g., a hole in the transverse connector 120), which maintains alignment of the longitudinal adjuster 418 relative to the frame 404. In some embodiments, movement of the carrier 410 is via a ratchet mechanism. In some embodiments, movement of the carrier 410 is via a screw mechanism which can be fastened or released gradually to place the carrier 410 at a selected position. This may provide for a more precise adjustment of the axial position of the carrier 410 than by merely adjusting the elongate members 416.

In some embodiments, as shown for example in FIG. 6, one or more wires 448 are passed through holes 432 formed in the fixator member(s) 408. (In this example, a wire 448 is passed through two opposing holes 432 in a same fixator member 408. Additionally or alternatively, a single wire 448 can be passed through holes 432 of two separate fixator members 408). In embodiments in which two or more wires 448 are passed through the same fixator member 408, one or more slots formed on the fixator member may guide the different wires 448 so that each wire 448 crosses the fixator member 408 on a different (axial) plane, preventing the wires 448 from interfering with each other (such as by crossing each other at the same spatial point). In an example, two wires 448 extending through a same fixator member 408 are guided to lie on planes that are parallel to each other.

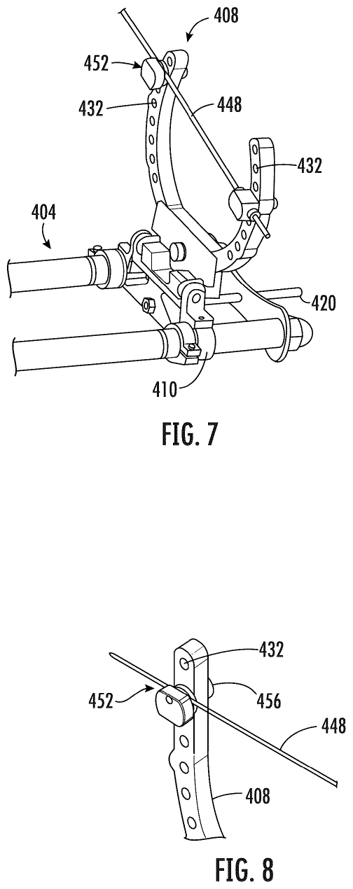

FIGS. 7-11 show wires 448 guided through holes 432 of one of the fixator members 408 of the fixation device 400, according to some embodiments. In the configurations illustrated in FIGS. 6-11, the wires 448 are coupled to the holes 432 via holders 452.

In some embodiments, as shown in the various arrangements of FIG. 7, a wire 448 is coupled to the fixator member 408 at selected holes 432 of the fixator member 408. In some embodiments, the holes 432 configured to receive the ends of a particular wire 448 are located on diametrically opposing sides of the fixator member 408. For example, the wire 448 is passed through holders 452 (e.g. clamps), positioned at each 432. In some embodiments, the wire 448 is secured to the holder 452, for example using a fixture 456 (e.g. a bolt, a clamp). Optionally, rotation of the fixture 456 (such as within the holder 452 or at the hole 432) increases tension on the wire 448. FIG. 8 illustrates a detail view of the wire 448 secured to the fixator member 408 via the holder 452. In some aspects, a distance between the holder 452 and the fixator member 408 can be adjusted to prevent wires 448 secured to the same fixator member 408 from intersecting with each other.

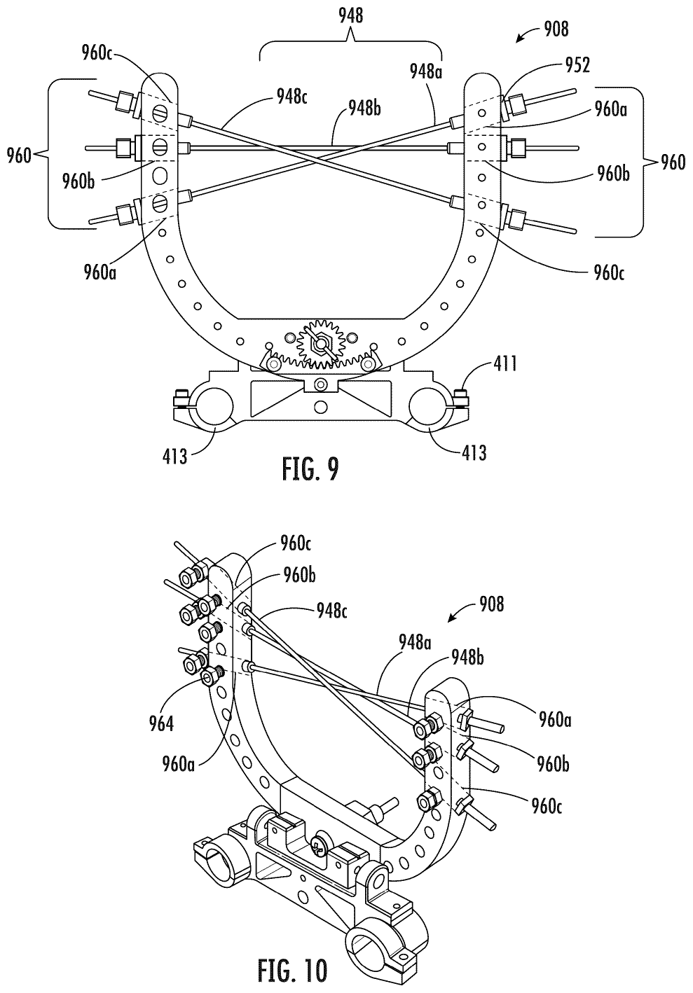

FIGS. 9-11 illustrate an arrangement for preventing a plurality of wires 448 coupled to the same fixator member 908 from intersecting with each other. The fixator members 908 are substantially the same as the fixator member 408 and are only described in detail to the extent that they differ from the fixator members 408. In some embodiments, the fixator member 908 comprises a plurality of slots 960 configured to receive the holders 952, where each slot 960 extends across a thickness TB of the fixator member 408 (such as in a perpendicular alignment relative to a longitudinal axis B (FIG. 5) of the device frame 404). The slots 960 are typically arranged in pairs configured to receive first and second ends of the wire 948. For example, FIGS. 9-11 illustrate an embodiment that that includes three pairs of slots, slots 960a, 960b, and 960c, configured to receive three different wires, 948a, 948b, 948c, respectively. Other embodiments may include more or fewer slots 960 and/or wires 948.

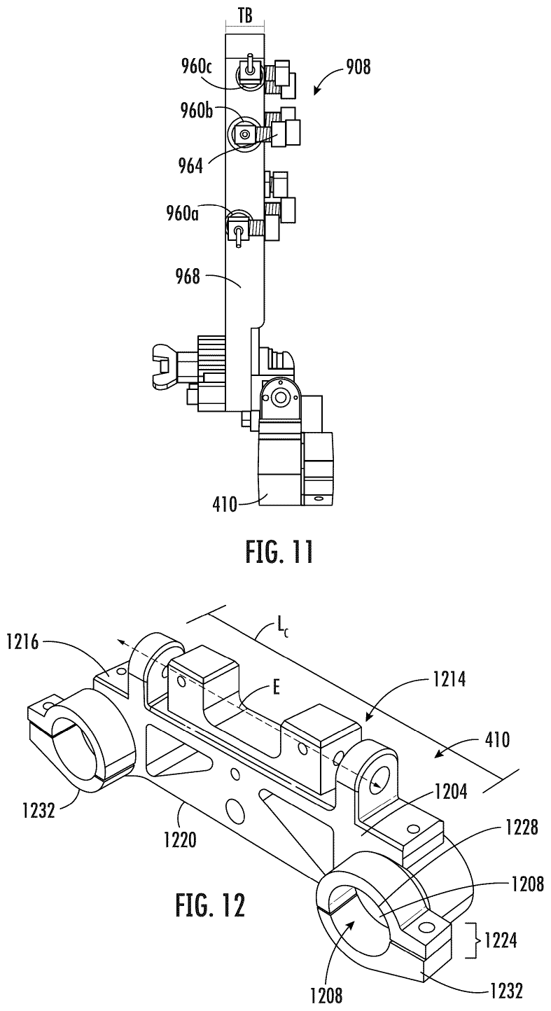

In such embodiments, as shown for example in FIGS. 10-11, the slots 460 are arranged in different positions along the thickness TB of the fixator member 408 such that, when two or more wires 448 are guided through a single fixator member 408, the slots 460 are arranged to guide the wires 448 along non-crossing pathways, for example, on pathways extending along parallel planes, to avoid collision of the wires 448, which may interfere with tensioning and the like. As shown in FIGS. 10-11, it is shown that the slots (along with the fastener 964 placed at each slot) are arranged in a non-linear manner relative to width (thickness) of the fixator member wall 968. As shown in FIGS. 10-11, the pairs of slots 960a, 960b, and 960c are positioned such that the wires 948a, 948b, and 948c, respectively, are spaced apart from each other and do not intersect.

In some embodiments, wires 944 are threaded through the slots 960 and are attached and fastened at to the slots 960, for example using a screw or other fastener 964. In some embodiments, the fasteners 964 are positioned within the slots 960.

FIGS. 12-15 illustrate various embodiments of the carrier 410 attachable to a fixation device frame, such as the frames 104, 404, according to some embodiments. In such embodiments, the carrier 410 is configured to couple the fixator member 408 to the frame 404. In some embodiments, the carrier 410 comprises a body 1204 defining holes 1208 configured to receive the elongate members 416 of the frame 104. In some embodiments, the carrier 410 is coupled to the frame 404 by inserting the elongate members 416 of the frame 410 through the holes 1208, and then sliding the carrier 410 to a selected position along the length of the frame 404 in which the fixator member 408 is to be secured. In some embodiments, movement of the carrier 410 along the length of the frame 404 is controlled by a twisting screw mechanism such as the longitudinal adjuster 418.

In some embodiments, the carrier 410 is sized to extend transversely between the elongate members 416 of the frame 404. In such embodiments, a length Lc of the carrier 410 is similar to the width of the frame 404. In some embodiments, a length Lc (FIG. 12) of the carrier 410 may be adjustable to change a distance between the elongate members 416 of the frame 404. In other embodiments, carriers 410 may be provided in a variety of lengths Lc such that the width of the frame 404 (e.g., distance between the elongate members 416) can be determined based on a size of the limb being treated. In such embodiments, a length of the transverse connector 120 may also be adjustable and/or transverse connectors 120 of varying lengths may be provided.

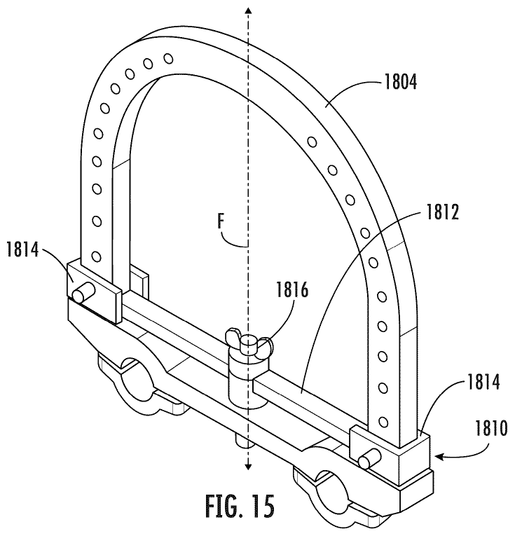

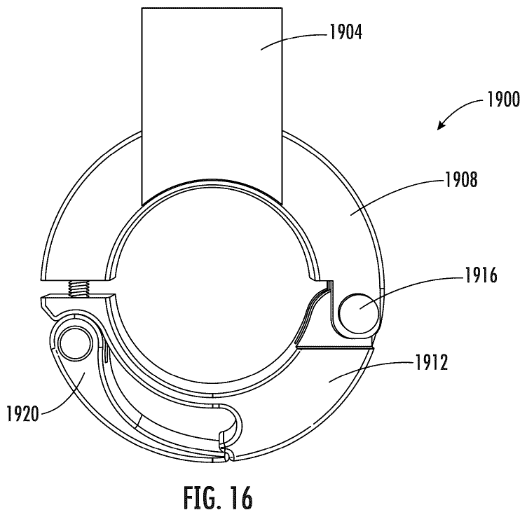

In some embodiments, the carrier 410 comprises holders 1214 configured to be coupled to a fixator member 408 (FIGS. 13, 15). In the configuration illustrated in FIG. 12, the holders 1214 include slots configured to receive ends of the fixator member 408. Fasteners (not shown) may be used to secure the ends of the fixator member 408 within the holders 1214. In some embodiments, the fasteners may be configured to allow rotation of the fixator member 408 relative to the frame 404 when the fixator member 408 is coupled to the holders 1214. In other embodiments, the holders 1214 may have other configurations, such as the coupling plate 414, a clamp, a recess, and so forth. Optionally, the holders 1214 are located on an upper portion 1216 of the carrier 410, facing upwards so as to hold the fixator member 408 above the frame 404, for example, in a vertical alignment relative to the frame 404.

In some embodiments, the carrier 410 may include a rotation mechanism 1604 fixator member 408 about an axis E towards the device frame 104, 404 (e.g., into the page).

FIG. 13 illustrates the carrier 410 which coupled to a fixator member 408 in a configuration in which the fixator member 408 extends upward from the frame 404, according to some embodiments. In some embodiments, the carrier 410 is slidable along the device frame 404 such that the fixator member 408 can be repositioned. For example, in some embodiments, the carrier 410 includes a first surface 1216 including the holders 1212 configured to engage the fixator member 408 and a second, opposite, surface 1220 including attachment features 1224 configured to releasably engage the elongate members 416.

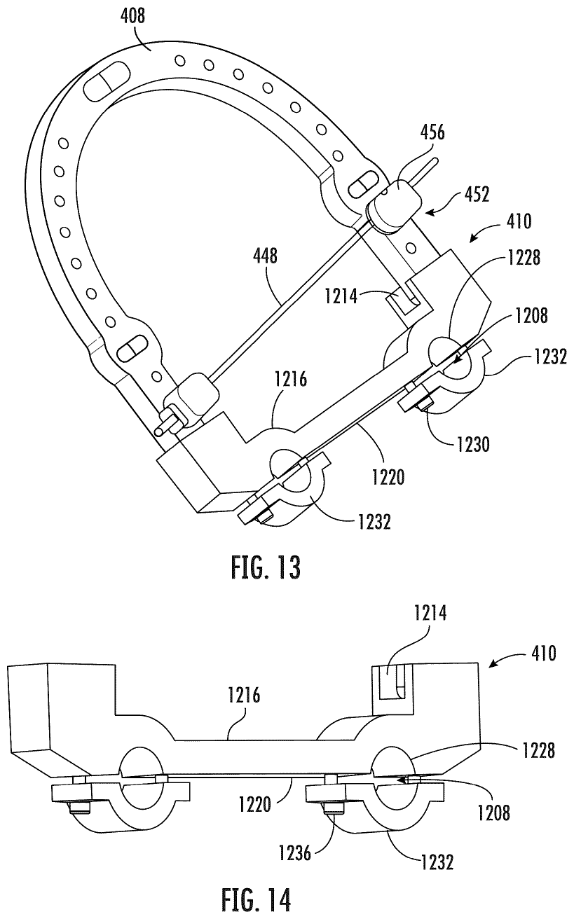

As is best shown in in FIG. 14, the attachment features 1224 include recesses 1228 formed between the second surface 1220 of the carrier 410 and removable clamping portions 1232 of the carrier 410. The recesses 1228 and the clamping portions 1232 cooperatively define the hole 1208 therebetween. In such embodiments, the elongate members 416 are positioned within the holes 1208 defined between the recesses 1228 and the clamping portions 1232 in a first configuration in which the carrier 410 can slide along the elongate members 416 and a second configuration in which the carrier 410 is fixedly secured to the elongate members 416. In other embodiments, the attachment features 1224 may have other configurations. The attachment features 1224 engage the elongate members 416 of the frame 404. In some embodiments, the clamping portions 1232 are coupled to the second surface 1220 of the carrier 410 by one or more screws 1236 which when fastened tighten the clamping portions 1232 to the second surface 1220 of the carrier 410, thereby securing a portion of the elongate member 416 within the hole 1208.

FIG. 15 shows a carrier 1810 configured to allow rotation of a fixator member 1804 about an axis F (for example being an axis perpendicular to a plane defined by the frame 1404, 404). The carrier 1810 and the fixator member 1804 are substantially similar to the carriers 110, 410, and fixator members 108, 408, respectively, and are only described in detail herein to the extent that they differ from the carriers 110, 410 and fixator members 108, 408. For example, in the embodiment illustrated in FIG. 15, the carrier 1810 does not include carriers configured to receive the fixator member 1804. Instead, the carrier 1810 is rotatably coupled to a bar 1812 that includes holders 1814 coupled to opposing ends of the bar 1812. In some embodiments, the holders 1814 may be substantially similar to the holders 1214.

The bar 1812 is coupled to the carrier 1810, for example via a fastener 1816. The bar 1812 has a first configuration in which the bar 1812 is rotatable relative to the carrier 1810 about the axis F and a second configuration in which the bar 1812 is fixed relative to the carrier 1810. For example, in the first configuration, the fastener 1816 is loosened, such that the bar 1812 can rotate relative to the carrier 1810. When the bar 1812 is in a desired position relative to the carrier 1810, the fastener 1816 is tightened, to secure the bar 1812 relative to the carrier 1810. Rotation of the bar 1812 allows the fixator member 1804 to be selectively rotated about axis F to a desired position, in which the fixator member 1804 may be at an offset angle relative to the carrier 1810.

In other embodiments, the fastener 1816 may be a multi-directional fastener, such as, for example a ball joint (e.g., with a locking screw). In some embodiments of the disclosure, the fastener 1816 and/or other hinges described herein may be operated using a motor, which may allow a user to avoid manipulating mechanical systems near the wounded limb. Alternatively or optionally, the fastener 1816 may be operated using an elongate tool, such as a screwdriver, a worm gear, and so forth . . .

FIG. 16 shows a quick release clamp 1900 which may be used in accordance with some embodiments of the disclosure. The clamp 1900 may be a quick release shaft color or toggle clamp. In the embodiment shown in FIG. 16, the clamp includes a base section 1904 that can be attached to or form part of a system component, such as a fixator member, carrier or other element to be coupled to the elongate members 116, 416 of the fixation device 100, 400. The clamp 1900 may be quickly attached or detached. In the embodiment shown, the clamp 1900 includes a first or top portion 1908 and a second or bottom section 1912. The first section 1908 is shaped to conform to the shape of the elongate members 116, 416. The second section 1912 is movable relative to the top section 1908 and is shaped to conform to the shape of the elongate members 116, 416. In the illustrated configuration, the first and second sections 1908 and 1912 are curved. In other embodiments, the first and second sections 1908, 1912 may have other shapes. In some embodiments, the first and second sections 1908, 1912 may be or include a resilient material such that the first and second sections 1908, 1912 may deform to conform to a shape of the elongate members 116, 416. In other embodiments of the disclosure, the first and second sections 1908, 1912 are not configured to deform to conform to the shape of the elongate members 116, 416 when they engage the tube/base. Although the first and second sections 1908, 1912 are shown and described as top and bottom sections, in other embodiments, the first and second sections 1908 and 1912 may be laterally positioned relative to each other, at least in part.

In some embodiments of the disclosure, the second section 1912 is coupled to the first portion 1908 a hinge 1916, such that the second portion 1912 can open enough to allow insertion of the elongate members 116, 416 between the first and second sections 1908, 1912. Optionally, a toggle lock 1920 is used to lock the second section 1912 to the top section 1908 with a portion of the elongate member 116, 416 therebetween. Optionally, the lock 1920 is adjustable, for example, using a screw.

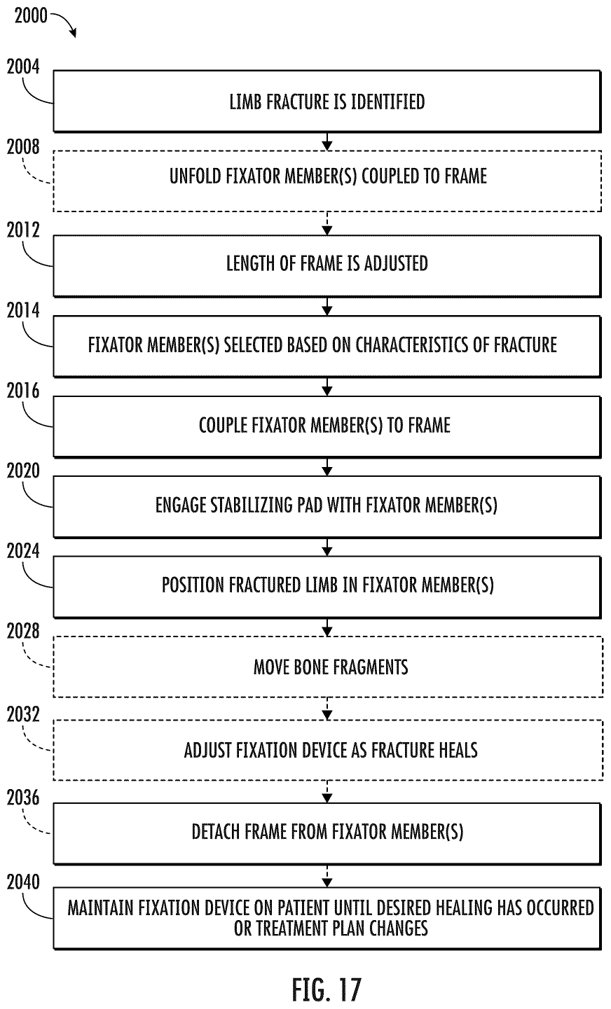

FIG. 17 illustrates a flowchart of a method 2000 for treating a limb fracture using a fixation device, such as the fixation device 100, 400, 3800 according to some embodiments.

At 2004, in some embodiments, a limb fracture is identified. In some embodiments, the fracture is identified (or suspected) immediately or close to its occurrence, for example, in an emergency situation. Examples of emergency situations may include a car accident, nature disasters (e.g. earthquake), battle field, and the like. The practitioner decides to perform damage control treatment, intended to prevent or reduce further fracturing of the limb, and optionally to initiate alignment and/or stabilization of the fracture to facilitate and/or accelerate healing. The treatment may occur at or near a location of the emergency situation or in a field hospital, etc. In some cases, the fixation device is used in a situation in which suitable surgical facilities, equipment and/or medical expertise are not immediately available, and damage control treatment needs to be performed. In some cases, fixation device is used when the patient's condition does not allow additional surgical intervention, for example so as to avoid further risk or damage (e.g. a “secondary hit”). For example, the patient may have extensive injuries and/or burns to the soft tissue near the fracture, so further disturbance of the soft tissue due to surgery is not possible. However, the fixation device 100, 400, 3800 may also be used in an operating room.

At 2008 (optional), in embodiments in which the device 100, 400, 3800 is provided in a folded, collapsed state, the device 100, 400, 3800 is unfolded. In some embodiments, unfolding is performed manually. In some embodiments, the device 100, 400, 3800 (such as a frame 104, 404, 3804 of the device 100, 400, 3800) may include clastic components (e.g. a spring) which facilitate device transformation from a collapsed state to an open, deployable state.

At 2012 (optional), the size (e.g. length, width) of the frame 104, 404, 3804 is adjusted. For example, the length of the frame 104, 108 may be adjusted to fit a length of the limb to be treated for a specific patient, for example by pulling on or pushing on one or both ends of the frame 104, 404, 3804 to adjust a length of the elongate members 116, 416, 4816. Additionally or alternatively, extensions 128 may be coupled to the elongate members 116, 416, 4816 to increase a length of the frame 104, 404, and/or two or more elongate members 116, 416, 4816 can be connected to each other utilizing bridging connectors, to adjust a total length of the frame 104, 404, 3804. For example, if the fracture is a tibial fracture, the length of the frame 104, 404, 3804 is adjusted to match an axial length of the tibia of the patient (such as from below the knee to the ankle of the patient). In another example, if the fracture is peri-articular (e.g., in the vicinity of a joint such as the ankle, knee, hip, elbow, shoulder joints), the length of the frame 104, 404, 3804 can be adjusted such that one of the carriers 110, 410, 3810 can be positioned above the affected joint on a first side of the fracture and another of the carriers 110, 410, 3810 can be positioned below the affected joint on a second side of the fracture (or a more distal portion of the fractured limb), such that the frame 104, 404, 3804 spans the fracture involving the affected joint.

At 2014, a particular shape or type of fixator member 108, 408, 3808 to be used at particular positions along the frame 104, 404, 3804 is selected based on characteristics of the fracture. Characteristics of the fracture may include location of fracture, type of fracture, condition of surrounding soft tissue, and so forth. Various types of fixator members may be provided, including for example U-shaped fixator members, L-shaped fixator members, arc shaped fixator members, ring shaped fixator members, partial ring shaped fixator members, bow shaped fixator members, horseshoe shaped fixator members, half-horseshoe fixator members, reverse omega-shaped fixator members, and/or posts.

At 2016 one or more fixator members 108, 408, 3808 may be coupled to the frame 104, 404, 3804. Further, at 2016, an axial position of the fixator members 108, 408, 3808 along the frame 104, 404, 3804 may be adjusted by repositioning the carriers 110, 410, 3810. The practitioner arranges the fixator members 108, 408, 3808 along the frame 104, 404, 3804 based on the type and/or location of the fracture being treated. For example, the practitioner may position or reposition the carriers 110, 410, 3810 to establish the distance between adjacent fixator members 108, 408, 3808. The practitioner may position or reposition the carriers 110, 410, 3810 to establish the distance between the most proximal fixator member 108a, 408a, 3808a and the most distal fixator member. In some embodiments, the practitioner may position or reposition a rotational orientation of the fixator members 108, 408, 3808 relative to the carriers 110, 410, 3810.

At 2020, the stabilizing pad 112, 3812 may be engaged with the fixator members 108, 408, 3808, the carriers 110, 410, 3810 and/or the frame 104, 404, 3804, at either of steps 2008, 2012, and/or 2016.

At 2024, the practitioner positions the fractured portion of the limb in the deployed device 100, 400, 3804. In some embodiments, positioning the fractured limb includes resting the limb against the stabilizing pad 112, 3812 coupled to the device frame 104, 404, 3804, the carriers 110, 410, 3810, and/or the fixator members 108, 408, 3808. For example, positioning the limb includes introducing the limb into an elongate spacing defined between the fixator members 108, 408, 3808 and the stabilizing pad 112, 3812. In some embodiments, the limb portion is positioned such that the limb portion is supported by the fixation device 100, 400, 3800, and the fixation device 100, 400, 3800 immobilizes the limb portion. The stabilizing pad 112, 3812, which is positioned between the limb portion and the device frame 104, 404, 3804 and/or the fixator members 108, 408, 3808, at least partially restricts movement of the limb portion. For example, the stabilizing pad 112, 3812 may restrict lateral and/or longitudinal movement of the limb when the limb is supported by the stabilizing pad 112, 3812. This can allow a single practitioner to immobilize the limb without assistance. The stabilizing pad 112, 3812 suspends the limb portion the spacing S above the frame 103, 404, 3804 and/or the fixator members 108, 408, 3808. In some embodiments, the thickness of the stabilizing pads 112, 3812 is selected according to the limb anatomy. Optionally or additionally, the type of stabilizing pad 112, 3812 is selected according to the wound (e.g., different materials for open and closed fractures and/or for burns. In some embodiments of the disclosure, no padding may be is placed at least at some axial locations where there are fixator members 104, 404, 3804 and the limb is stabilized solely using wires and/or pins, at least at those axial locations.

Therefore, an advantage of having the fractured portion of the limb rest against the fixation device 100, 400, 3800 (e.g. against the stabilizing pad 112, 3812) is that the device 100, 400, 3800 forms a workstation or hub on which the limb is immobilized and the fixation device 100, 400, 3800 can act as a mini fracture table. This facilitates access to the limb, reduction of the fracture, and can allow the fixation process to be carried out by a single practitioner (e.g., for example additional hands to hold the limb in place during the process may not be required). Further, the stabilization provided by the device 100, 400, 3800 may allow fixation to be performed more accurately, potentially reducing the risk of damage to surrounding tissues. Further, the device 100, 400, 3800 can stabilize the limb during imaging (such as during an X-ray), potentially reducing pain to the patient and/or reducing the risk of damage to the soft tissues of the limb and/or the bone, for example due to uncontrolled movement of the limb.

At 2024, the practitioner introduces one or more wires 448, 3848 through the bone, and/or pins into the bone. In some embodiments, insertion of the wires 448, 3848 and/or pins is guided by the fixation device 100, 400, 3800. For example, in some embodiments, the fixator members 108, 408, 3808 are arranged (e.g. by the practitioner) such that a wire 448, 3808 or a pin that is guided through holes 432, 3860 of either of the same fixator member 108, 408, 3808 or of adjacent fixator members 108, 408, 3808 is directed to pass through the limb portion in an anatomical path selected to provide for reconstruction of the bone fragments (e.g. by tensioning a wire between bone fragments to approximate them towards each other) and to the least damage to the surroundings soft tissues, such as muscles, blood vessels, nerves. In some embodiments, the selective arrangement of the fixator members is performed based on the type and/or severity and/or specific anatomical location of the bone fracture.

For example, the fixator member 108, 408, 3808 may include a plurality of holes 432, 3860 through which the wires 448, 3848 and/or pins are passed. In embodiments in which wire 448, 3848 is used, the wire 448, 3848 may guided through a selected hole 432, 3860 in the fixator member 108, 408, 3808 and into and through the bone, and then guided through a different hole 432, 3860 in the same fixator member 108, 408, 3808 or a hole 432, 3860 formed in a different (e.g. adjacent) fixator member 108, 408, 3804. The ends of the wires 448, 3848 are then secured in the holes 432, 3860. In some embodiments, the inserted wires 448 and/or pins are secured to the fixator member 108, 408, 3808 either directly or via the holder 452. In embodiments that include the holder 452, the holder 452 is coupled to the fixator member 108, 408 (e.g., via the holes 432). In some embodiments, the holder 452 defines a lumen or a hole through which the wires 448 or pin is passed. In such embodiments, the wires 448 and/or pins are guided through the holder 452. In some embodiments, a holder is configured to be moved along the fixator member, for example slid along the fixator member, and then stopped or locked at a desired position along the fixator member. In some embodiments, hold of the bone fragments is obtained using the inserted wires 448, 3848 and/or pins. In some embodiments, each bone fragment is engaged by at least one wire 448, 3848 and/or by at least one pin.

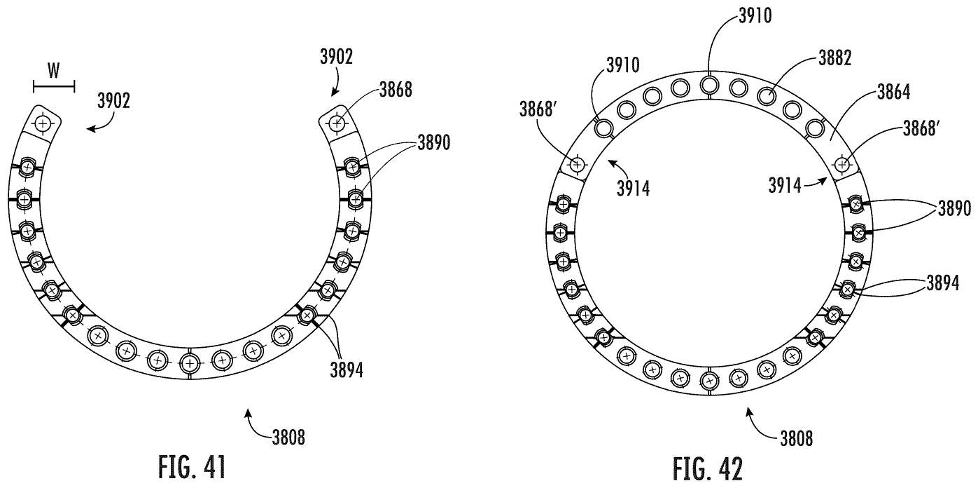

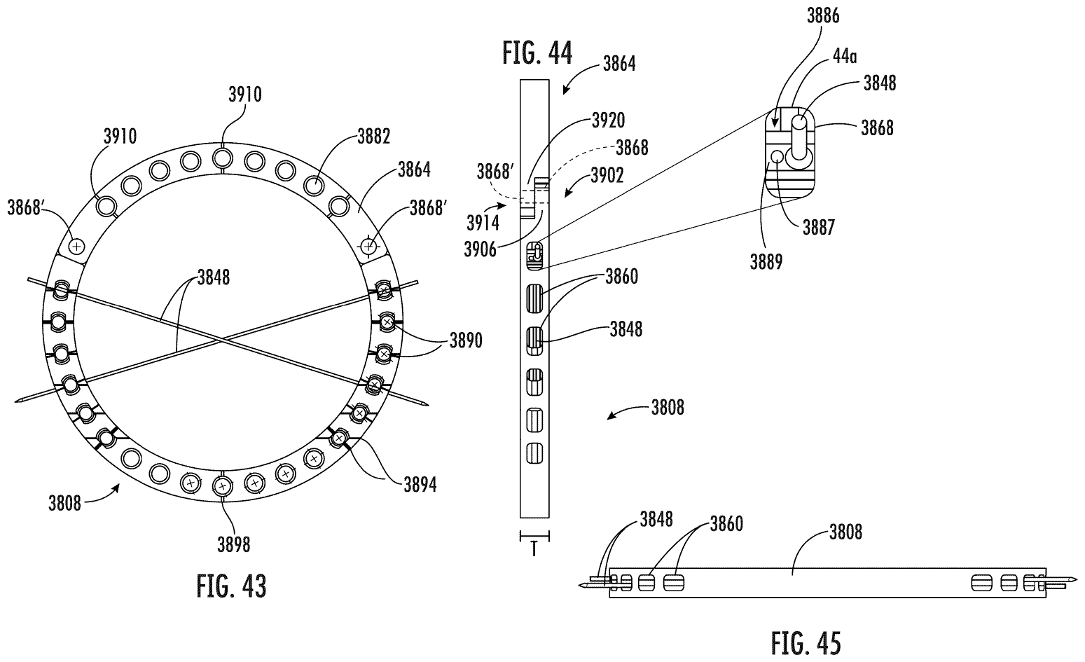

In some embodiments, the hole(s) 432, 3860 in the fixator member 108, 408, 3808 and/or hole(s) in the holder 453 are marked to facilitate identifying the path for guiding the wire 448, 3848 and/or pin through (FIGS. 41-43). For example, in some embodiments, the holes 432, 3860 may be numbered, colored (e.g. around the hole), or otherwise indexed. In such embodiments, two holes 432, 3860 (either on the same fixator member 108, 408, 3808 or on two adjacent fixator members 108, 408, 3808 and/or on holders 452 coupled to a fixator member 108, 408, 3808) are marked with a similar marking, indicating that a wire 448, 3848 is to be passed through (and optionally secured to) both holes 432, 3860. In other embodiments, the holes 432, 3860 may include markings indicating a path for wires 448, 3848 positioned through acceptable pairs of holes 432, 3860.

In some embodiments, wires 448, 3848 and/or pins are introduced to the limb using the proximal 108a, 408a, 3808a and distal fixator members 108b, 408b, 3808b of the device first, and then additional wires 448, 3848 and/or pins are introduced to the limb using one or more intermediate fixator members 108, 408, 3808, such as fixator members 108, 408, 3808 positioned between the proximal 108a, 408a, 3808a and distal fixator members 108b, 108b, 3808b that were added onto the frame 104, 404, 3804 at 2016. In the illustrated embodiments, the intermediate fixator members 108, 408, 3808 are shown coupled to the frame 104, 404, 3808 such that an opening of the fixator members 108, 408, 3808 faces away from the frame 104, 404, 3804. In other embodiments, the intermediate fixator members 108, 408, 3808 may be coupled to the frame 104, 404, 3808 such that an opening of the fixator members 108, 408, 3808 faces toward the frame 104, 404, 3804.

In some embodiments, the wires 448, 3848 and/or pins are configured to penetrate through the skin layer, muscle tissue and bone, while the limb is stabilized by the fixation device 100, 400, 3800 with or without the stabilizing pad 112, 3816.

In some embodiments, at 2024, the wires 448, 3848 may first be coupled between the distal portion of the patient's limb (e.g., proximate the ankle for a tibia fracture) to the distal fixator member 108b, 408b, 3808b. The wires 448, 3848 next may be coupled between the proximal portion of the patient's limb (e.g., proximate the knee for a tibia fracture) to the proximal fixator member 108a, 408a, 3808a. At this point, the patient's limb is suspended in the fixation device 100, 400, 3800 and has been stabilized.

At 2028, which is optional, the bone fragments are moved. Movement of the bone fragments may be performed, for example, manually and/or using the wires 448, 3848 and/or pins that were inserted to hold the bone fragments. In some embodiments, movement of the bone fragments is performed to place the fragments at a desired position using the fixation device 100, 400, 3800 (including, for example, to set an axial and/or transverse and/or rotational alignment between the fragments; to set a rotational orientation; and/or otherwise define a position of the fragments relative to each other and/or relative to portion(s) of the same bone or other bone(s)). For example, in some embodiments, tension is applied to the inserted wires 448, 3848 and/or pins, for example to apply the desired force(s) on the bone which are needed, for example, to approximate bone fragments towards each other and/or otherwise set a position and/or alignment of the bone fragments, e.g., relative to each other and/or the fixation device 100, 400, 3800.

In embodiments in which fixator members 2500, 2700 (FIGS. 22-24) having rotary portions 2504, 2704 are used, the distal fixator members 108b, 408b, 3808b may be rotated to better align the bone fragments.

At 2032, which is optional, adjustments are made to the device 100, 400, 3800 while the device 100, 400, 3800 deployed is on the limb, for example, by tensioning the wires 448, 3848, re-arranging a position and/or orientation of the fixator member(s) 108, 408, 3808, modifying a length of the frame 104, 404, 3804, and/or other adjustments.

At 2036, which is optional, the frame 104, 404, 3804 may be detached from the fixator members 108, 408, 3808, such that the fixator members 108, 408, 3804 remain coupled to the patient's limb. In some embodiments, the limb may be stabilized without relying on imaging.

At 2032, the fixation device 100, 400, 3800 is maintained on the limb until a desired reduction or healing of the fracture has occurred, and/or a decision has been made to change the type of treatment provided to the patient.

In some embodiments, the fixation device 100, 400, 3800 is maintained on the patient until a surgical procedure is performed. In some embodiments, the fixation device 100, 400, 3800 is maintained on the patient until deciding to change to a different type of fixation, for example based on imaging results. In some embodiments, the fixation device 100, 400, 3800 is maintained on the patient and is optionally reconfigured, such as by adjustment of components as the frame 104, 404, 3804 and/or the fixator members 108, 408, 3808, to fit for long term and optionally definitive fixation. Once fixator members 108, 408, 3808 are in place (and optionally, fixed relative to one another, frame 104, 404, 3804 may be removed altogether, thereby leaving fixator members 108, 408, 3808 affixed to the patient's limb.

In some embodiments, at 2024, the wires 448, 3848 and/or pins are introduced into (or through) the limb portion using the holes 432, 3860 of the fixator member 108, 408, 3808 without performing imaging. In such embodiments, the holes 432, 3860 lead the wires 448, 3848 and/or pins via paths along which damage to surrounding tissue is reduced or prevented. A potential advantage of the ability to deploy the fixation device without performing imaging prior to and/or during the insertion of wires 448, 3848 and/or pins may include that the device can be used immediately “on site”, even in field conditions or the like when no sufficiently-equipped clinic or field hospital facilities are available.

In some embodiments, imaging (for example, an X-ray scan) of the fractured limb is performed at a later stage, for example, when imaging is made available.

In an example of use, when treating a tibial fracture, a proximal fixator member 108a, 408a, 3808a may be placed above the knee, at the distal femur. The fixator member 108a, 408a, 3808a may include a hole 432, 3860 marked according to the desired placing of the wire 448, 3848, for example RF1-right femur, wire number 1. When treating, for example, a contralateral limb fracture, a similarly placed fixator member 108, 408, 3808 may be used, having another hole marked, for example, LF1-left femur, wire number 1.

In a similar manner, for a fixator member 108, 408, 3808 placed at the proximal tibia, the fixator member 108, 408, 3808, may include a hole marked RT1-right tibia, wire number 1, for treating a tibial fracture; and another hole marked LT1-left tibia, wire number 1, for treating a contralateral limb fracture.

If an additional wire 448, 3848 is needed, a hole 432, 3860 for that wire 448, 2848 may be marked RF2 (or LF2) and so forth.

In some embodiments, a distal fixator member 108b, 408b, 3808b, may include markings in a similar manner.

In one example, a fixator member 108, 408, 3808 may have color-coded locations. For example, for a first type of fracture one color is used to mark the holes 432, 3860 and for another type of fracture or treatment, a different color is used. Alternatively or optionally, different wires 448, 3848 have different colors, color coded for use and/or for fracture identification. For example, a color of a particular wire 448, 3848 may have the same color as a particular hole or pair of holes 432, 3860.

A potential advantage of the guiding a wire 448, 3848 through the designated, marked holes 432, 3860 may include that the wire 448, 3848 is passed along a “safe path” of the limb, where contact with blood vessels and/or nerves is avoided as much as possible.

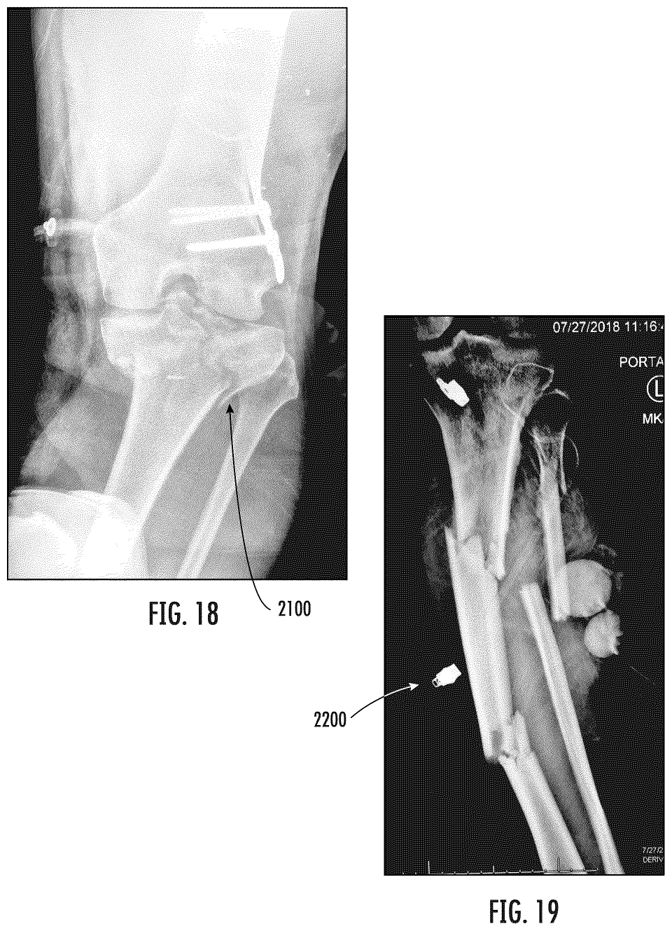

FIGS. 18-19 illustrate exemplary fractures that can be treated using the fixation device 100, 400, 3800 according to embodiments of the present disclosure. FIG. 18 illustrates a tibial plateau fracture 2100. FIG. 19 illustrates a diaphyseal tibial shaft fracture 2200.

The fixation devices 100, 400, 3800 described herein are suitable for treating long bone fractures, including a shaft of the long bone (FIG. 18) and/or peri-articular fractures (FIG. 19) including proximal or distal parts of the long bone. As used herein, the phrase “long bone” refers the tibia, femur, humerus, radius, ulna. In some embodiments, the fixation device is suitable for treating fractures closer to the pelvis or at the pelvis, such as proximal femur fractures, pelvis fractures. Optionally, when using for other limbs and/or for the pelvis, the shape (e.g., relative width) and/or size of individual fixator elements and/or the base is modified to accommodate the larger (or smaller) body parts and/or their different shapes.

In some embodiments, the fixation device 100, 400, 3800 may be used for immediate treatment of severe injures such as open fractures. Open fractures are often associated with injury of soft tissue surrounding the bones, and when fixating and/or otherwise treating the fracture, special care needs to be made not to further damage the surrounding soft tissue.

In some embodiments, different types of fractures are treated by different arrangements and/or different positioning of the fixation device 100, 400, 3800 relative to the patient anatomy. In the fracture 2100 shown in FIG. 18 for example, the fixation device 100, 400, 3800 may be arranged so that one or more wires 448, 3848 or one or more half pins are guided through a most proximal fixator member 108a, 408a, 3808a of the device 100, 400, 3800 and into a distal portion of the femur, and another one or more wires or half pins are guided through a most distal fixator member 108, 408, 3808 of the device 100, 400, 3808 into the tibia or the calcaneus. In some embodiments, wires 448, 3848 and/or half-pins are guided from the pelvis down to the distal femur, tibia or calcaneus.

In some embodiments, for treating a fracture along the tibial shaft for example, wires 448, 3848 and/or half pins are guided into proximal and/or distal portions of the tibia, and/or into the distal femur, and/or into the calcaneus. In some cases it is preferred to pass wires 448, 3848 and/or half pins through the femur and the calcaneus to avoid passing them directly through the fractured tibia, for example to reduce a risk of contamination (e.g. of the intramedullary canal). Passing wires 448, 3848 and/or pins in a path that avoids the fractured tibia (or generally any fractured bone that is treated using the fixation device) may also be advantageous if a second procedure takes place, such as introducing of an intramedullary nail or a plate into the fractured tibia.

In some embodiments, one or more additional fixator members 108, 408, 3808 are attached to the device frame 104, 404, 3804 and wires 448, 3848 and/or pins are guided through the fixator members 108, 408, 3808. Optionally, the one or more additional fixator members 108, 408, 3808 are positioned between the proximal fixator members 108a, 408a, 3808a and distal fixator members 108b, 408b, 3808b (which, in some embodiments, are integrally attached to the frame 104, 404, 3804). A potential advantage of selectively positioning fixator member(s) 108, 408, 3808 along the frame 104, 404, 3804 at desired distance(s) from other fixator members 108, 408, 3808 may include optimizing the type of fixation required for a specific fracture and/or a specific patient anatomy, for example by selecting a distance between adjacent fixator members 108, 408, 3808 such that the distance between the adjacent fixator members 108, 408, 3808 provides a desired reduction of the fracture and/or other desired displacement of the fracture fragments.

In some embodiments, the fixation device 100, 400, 3800 is configured to provide for initial reconstruction of the broken bone by enabling alignment of the bone fragments along: a long axis of the bone (such as by distancing or approximating the fixator members through which wires and/or pins are passed); a wide (transverse) axis of the bone (such as by adjusting or selecting a width of the frame, adjusting or selecting a width of a fixator member or using two half fixator members or using a tapering fixator member and selecting fixation in a section with the desired width); a rotational alignment, such as by rotating a fixator member relative to the frame such that a plane on which the fixator member lies on is not necessarily perpendicular to a plane defined by the frame (for example, a plane defined by the fixator member is at an angle of between 1-89 degrees to a plane defined by the frame); a vertical alignment, such as by setting a position of the wires 448, 3848 and/or half pins relative to the fixator member(s) 108, 408 and/or the frame 104, 404, 3804 or by attaching the fixator member 108, 408, 3808 to the frame 104, 108, 3808 using a screw or other variable length mechanism and setting the length of the mechanism as desired (e.g., FIG. 15, where the screw might be used for locking a rotational position and/or be used for setting a height). Optionally or additionally, the fixator member 108, 408, 3808 is attached to the frame 104, 408, 3808 or to the bar 1812 coupled to a carrier 1800 via a ball joint (or other multi-directional joint such as a universal joint), which ball joint is lockable, for example, using a pressure screw against it at a desired orientation.

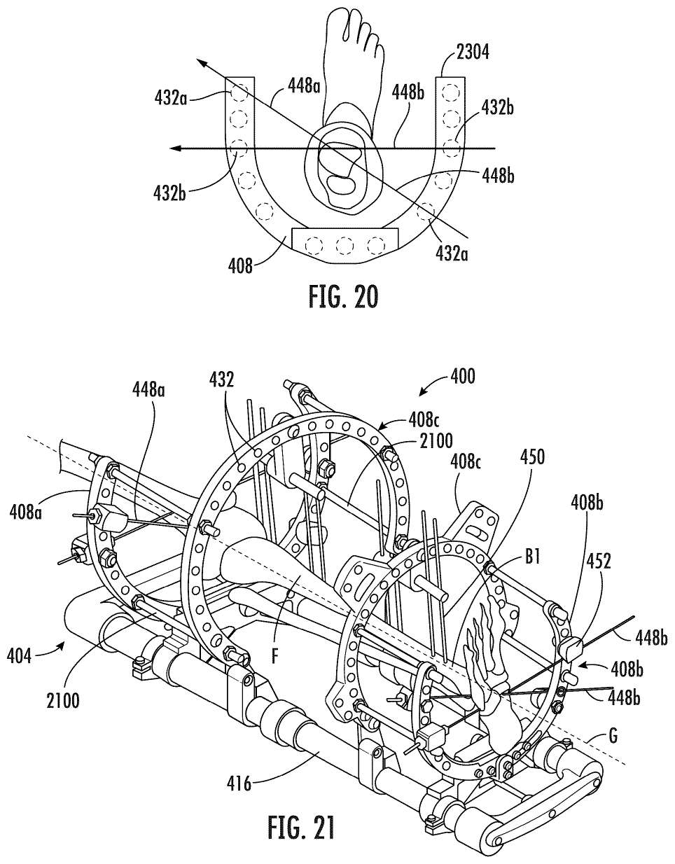

FIGS. 20-21 illustrate schematic views of wiring placed through bone(s) received within a fixation device, such as the fixation devices 100, 400 according to some embodiments.

FIG. 20 schematically illustrates a cross-sectional view of a configuration in which wires 448a, 448b are coupled to bone fragments by guiding each of the wires 448 through a pair of holes 432 formed in the fixator member 408. For example, the wire 448a extends through the pair of holes 432a and the wire 448b extends through the pair of holes 432b. In the configuration illustrated in FIG. 20, each of the wires 448a, 448b passes through a different bone fragment of a different portion of the bone.

In some embodiments, the paths for the wires 448 (e.g., per limb and/or disorder) are selected so that no or least no vital neurovascular structures are present along the path, potentially enabling introducing of the wires without imaging guidance. Optionally, the selection is pre-marked on the fixator members 108, 408 and/or in attached instructions or on an associated software. For example, FIG. 20 shows indicia 2304 indicating pairs holes 432 configured to receive the ends of the same wire 448. Such indicia 2304 may include matching pairs of digits, letters, graphics and/or colors on the fixator members 408. In some embodiments, the indicia 2304 may indicate which pairs of holes 432 may be associated with a particular axial limb location, fracture type, and/or wound type. In embodiments in which the indicia 2304 are intended for pins, the indica may not be paired. Further, in embodiments in which a particular hole 432 can be paired with different other holes 432, the particular hole may include multiple indicia 2304.

FIG. 21 illustrates a tibial fracture F being treated using a fixation device such as the fixation device 100, 400, according to some embodiments. Although the numbering shown in FIG. 21 is for the fixation device 400, the fixation device 100 can be deployed in a similar manner. In some embodiments, as shown in this example, one or more wires 448a are guided through the proximal fixator member 408a of the fixation device 400 into and through either the distal femur or proximal tibia. One or more additional wires 448b is guided through the distal fixator member 408b of the fixation device 400 into and through either the calcaneus or distal tibia. In some embodiments, as shown in this example, insertion of two more wires (e.g., the pairs of wires 448a and/or the pairs of wires 448b) through the same bone fragment is performed. Optionally, the pairs of wires 448 cross each other. A crossed arrangement of paired wires 448 potentially provides stability of the bone fragment along multiple axes and prevents bone slipping on wire 448 and/or may provide for setting a rotational orientation of the bone fragment (optionally in addition to an axial and/or transverse alignment). In some embodiments, crossing wires 448 are guided to pass on different planes, such as on parallel planes, so as to avoid intersecting with each other. In some embodiments, the designated holes 432 in the fixator member 108, 408 (and/or holders 452 or other wire holders located at the holes 432) are arranged to guide each of the wires to lie on a different plane (FIGS. 10-11). In one example, a set of holders 452 having different depths (e.g., extent away from the frame 104, 404) (for the wires/pins) is provided. Optionally, the axial separation between wires 448 (or wire centers) that cross each is between 1 and 20 mm, for example, between 2 and 10 mm, when the wires 448 are mounted on a fixator member 108, 408.

In some embodiments, as shown in FIG. 21, one or more additional fixator members 408 are attached to the device frame 104, 404, for guiding additional wires 448 and/or pins through the bone.

For example, as shown in FIG. 21, pins 450 are introduced through a bone fragment B1 such that the pins 50 lay on a plane that is substantially perpendicular to the long axis G of the fractured bone fragment B1. In some embodiments, pins may be introduced from the lateral to medial direction or from the medial to lateral direction of the bone.

In some embodiments, the wire 448 is rigid enough so as not to be deflected upon insertion. For example, the wire 448 may not be deflected by the hard bone cortex. In some embodiments, the wire 448 is thick enough so that when pushed through the limb, the wire 448 will not deflect from a linear path, which enables the wire 448 to reach, for example, the hole 432 on the other (exit) side of the limb. Optionally, a diameter of the wire is between 1.5 mm-3 mm, such as 2 mm or an intermediate, larger or smaller diameter. . . . For example, a .3 mm wire may be stiff and a 1.6 mm wire may be deflectable. Optionally, for deflectable wires, a holder 452 is used rather than the hole 432 in the fixator member 408. In some embodiments of the disclosure, the wire 448 is inserted using a drill and/or includes a drilling tip, so when that rotated by drill driver, the wire 448 drills into bone. Optionally, the holder 452 and/or indicia on the fixator member 408 is used as a guide for the positioning of the wire 448 during insertion. In other embodiments, a marking on the skin may be used as a guide for positioning the wire 448. In some embodiments of the disclosure, if the wire 448 is rigid enough, the wire 448 may be treated as a pin (e.g., may attach to the fixator member 408 on one side of the limb and may terminate in the body). For some limb sections and applications, a 2.5 mm diameter stainless steel, cobalt, or chrome wire 448 may be treated as a pin.

In some embodiments, one or more additional wires 448 and/or pins or half-pins may be introduced into the bone, for example to stabilize a bone fragment, or approximate bone fragments to each other, reducing a space between bone fragments and/or aligning bone fragments.

In some embodiments, the wire 448 guided through the bone is secured to the fixator member 408, for example by a fixation (e.g. a bolt, a tightening screw, the holder 452 for example as described herein). In some embodiments, the fixation is placed at the hole 432 through which the wire 448 is passed, or, in case a holder 452 (e.g. a clamp) is used and coupled to the hole 432. In some embodiments, the fixation allows for tightening of the wire 448, for example via manual (and/or tool-assisted) rotation. In some embodiments, the level of wire tension is increased (e.g., manually, e.g., to overcome creep) over time as the bone reconstruction process advances. In one example, such tightening is provided using a known method, such as using a Volkov-Oganesyan grooved screw or using two half pins at matching locations, that are rotatable to tension the wire 448.

In some embodiments, for example as shown in the example of FIG. 21, one or more fixator members 408 such as the proximal fixator member 408a and distal fixator member 408b are positioned so that an open portion 409 of the fixator member 408 faces upwards; while one or more other fixator members 408, such as 408c, are positioned such that a closed portion of the fixator member 408c faces upwards, optionally surrounding the bone. In some embodiments, (intermediate) fixator members 408c that are placed between the most proximal and most distal fixator members 408a, 408b, are configured for adjusting bone fragment displacement and/or for reinforcing stability of the fractured bone.

In some embodiments, for example when the fracture requires gradual reduction, one or both of the proximal fixator member 408a and distal fixator member 408b may be moved axially towards each other (e.g., via the longitudinal adjuster 418), to approximate the bone fragments towards each other. Alternatively, one or both of the proximal fixator member 408a and distal fixator member 408b may be moved axially away from each other in conditions in which bone fragments may be moved away from each other (e.g., via the longitudinal adjuster 418), for example when fragments overlap.

In some embodiments, once wires 448 and/or pins are inserted and, if needed, tightened, the stabilizing pad 112 of the fixation device 100, 400 (see for example FIGS. 1-2) can be removed, as shown for example in FIG. 21. In some embodiments, imaging of the limb portion is performed (it is noted that imaging may be performed with or without the stabilizing pad of the fixation device). In some embodiments, a material of the frame 104, 404, the fixator members 108, 408, the wires 448, the pins, and so forth may be suitable for use with a magnetic resonance imager (MRI). In such embodiments, the material of the frame 100, 400, the fixator members 108, 408, the wires 448, the pins, and so forth may be a non-magnetic material such as, for example, a composite material a plastic material, and/or non-ferromagnetic types of stainless steel.

In some aspects, adjacent fixator members 408 may be coupled together via one or more struts 2100. This may be done to reinforce the stiffness of the frame or in cases in which the frame 404 is removed. This also may be done to accommodate either future reduction of the displaced fracture fragments, or to facilitate better access to the injured extremity.

FIGS. 22-24 show configurations of fixator members 2500, 2700 that are configured to allow, rotation of a fixated limb, using a ring-in-ring design, in accordance with some embodiments of the disclosure. In some embodiments, the fixator member 2500 may be used as a distal-most fixator member when coupled to a frame such as the frames 104, 404. In some embodiments, the fixator member 2700 may be used as a proximal-most fixator member when coupled to a frame such as the frames 104, 404.

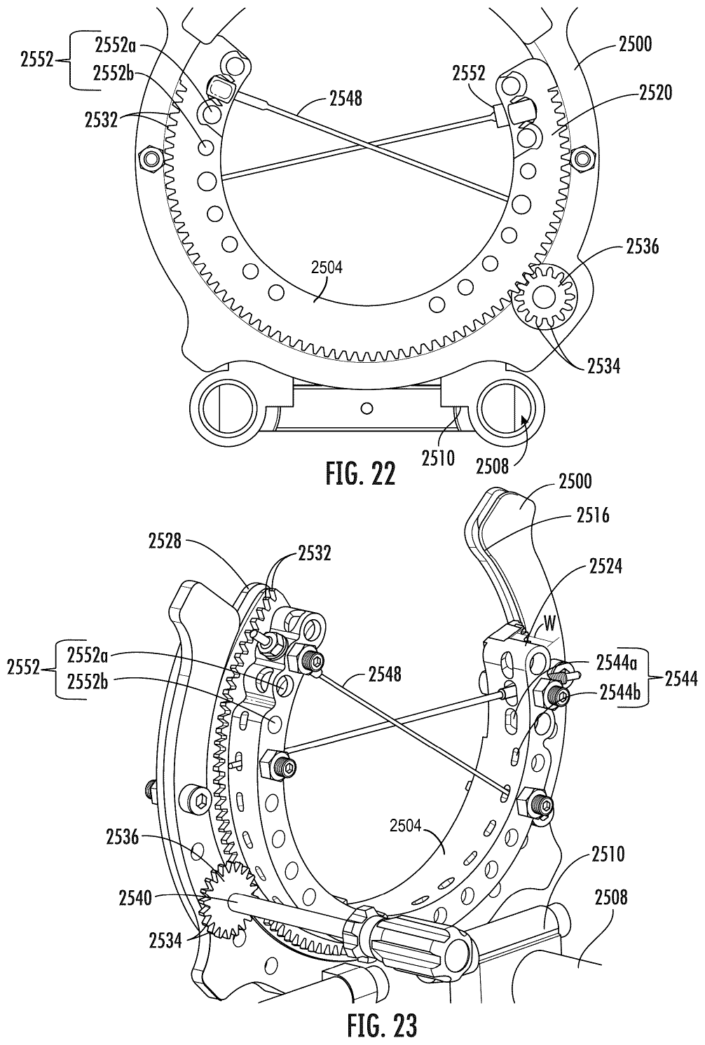

FIGS. 22-23 illustrate the fixator member 2500, which includes a rotary portion 2504 configured to rotate relative to the fixator member 2500. The fixator member 2500 is configured to be coupled to a frame 2508 via a carrier 2512. In the configuration illustrated in FIGS. 22-23, the fixator member 2500 is not configured to rotate relative to the frame 2508. For example, in the embodiment illustrated in FIGS. 22-23, the rotary portion 2504 is configured to rotate about a longitudinal axis of the frame 2508.

The fixator member 2500 further includes a track 2516 configured to receive a portion of the rotary portion 2504 therein. In the configuration illustrated in FIGS. 22-23, the track 2516 includes a groove. In other embodiments, the track 2516 may be another shape. In some embodiments, the track 2516 may extend along an entire inner circumference of the fixator member 2500. In other embodiments, the track 2516 may extend along one or more portions of the inner circumference of the fixator member 2500.

The frame 2508 is substantially similar to the frames 104, 404 and is not described in greater detail herein. The carrier 2512 is substantially similar to the carriers 110, 410 and is not described in greater detail herein.

The rotary portion 2504 includes a first portion 2520 and a second portion 2524 extending from the first portion 2520. In the illustrated embodiment, the second portion 2524 is stepped from the first portion 2520, such that a diameter of the first portion 2520 is larger than a diameter of the second portion 2524.

The first portion 2520 incudes a protrusion 2528 configured to be positioned within the track 2165. In some embodiments, the protrusion 2528 may extend along an entire outer circumference of the rotary portion 2504. In other embodiments, the protrusion 2528 may extend along one or more portions of the outer circumference of the rotary portion 2504.

The first portion 2520 further includes a plurality of teeth 2532 on the outer circumference of the first portion 2520. In some embodiments, the plurality of teeth 2532 may extend along an entire inner circumference of the fixator member 2500. In other embodiments, the plurality of teeth 2532 may extend along one or more portions of the outer circumference of the rotary portion 2504. In some embodiments, the plurality of teeth 2532 may extend along at least 90 degrees of the circumference of the first portion 2520. The plurality of teeth 2532 are configured to mesh with a plurality of teeth 2534 of an actuator gear 2536 coupled to the fixator member 2500. The actuator gear 2536 can be actuated (e.g., rotated) to reposition the rotary portion 2504. In the embodiment illustrated in FIGS. 22-23, the actuator gear 2536 is a pinion gear. In other embodiments, the actuator gear 2536 may have another configuration, such as a worm gear. In some embodiments, a tool 2540 or handle may be used to actuate the actuator gear 2536. In other embodiments, the actuator gear 2536 may be rotated by a motor. In some embodiments, the first portion 2520 and the fixator member 2500 may not include gears and/or teeth. In such embodiments, the gears and/or teeth may be replaced by detent mechanisms, a friction fit, and so forth.

With continued reference to FIGS. 22-23, the second portion 2524 of the rotary portion 2504 includes a plurality of holes 2544 along a circumference of the second portion 2524. The holes 2544 extend along a radial axis of the fixator member 2500. The holes 2544 are configured to receive wires 2548, pins, and/or clamps 2552 configured to receive wires 2548 and/or pins therein. In some embodiments, the holes 2544 are cylindrical in shape. In other embodiments, the holes 2544 may have other shapes. In some embodiments, the plurality of holes 2544 includes a first group of holes 2544a and a second group of holes 2544b. In some embodiments, the first group of holes 2544a are larger than the second group of holes 2544b. The first group of holes 2544a and the second group of holes 2544b may have different positions along a width W of the second portion 2524 such that wires 2548 positioned in the holes 2544a, 2544b do not intersect. For example, in the embodiment shown in FIGS. 22-23, each of the wires 2548 includes a first end positioned one of the first holes 2544a and a second end positioned in one of the second holes 2544b. This configuration causes the wires 2548 to be positioned in different planes such that they do not intersect.

In some embodiments, the rotary portion 2504 includes a plurality of holes 2552 that extend in a direction transverse to the holes 2544. For example, in the illustrated embodiment, the holes 2544 extend along a radial axis of the fixator member 2500 and the holes 2552 extend along an axis that is parallel to a longitudinal axis of the fixator member 2500. In some embodiments, the plurality of holes 2552 are configured to receive clamps (e.g., similar to the holders 452) and/or fasteners configured to secure the wires 2548 to the rotary portion 2504. In some embodiments, the holes 2552 are circular in shape. In other embodiments, the holes 2552 may have other shapes. In some embodiments, the plurality of holes 2552 include two groups of holes 2552a and 2552b. In the embodiment illustrated in FIGS. 22-23, the first group of holes 2552a is positioned proximate an open end of the rotary portion 2504. In some embodiments, the first group of holes 2552a is larger than the second group of holes 2552b.

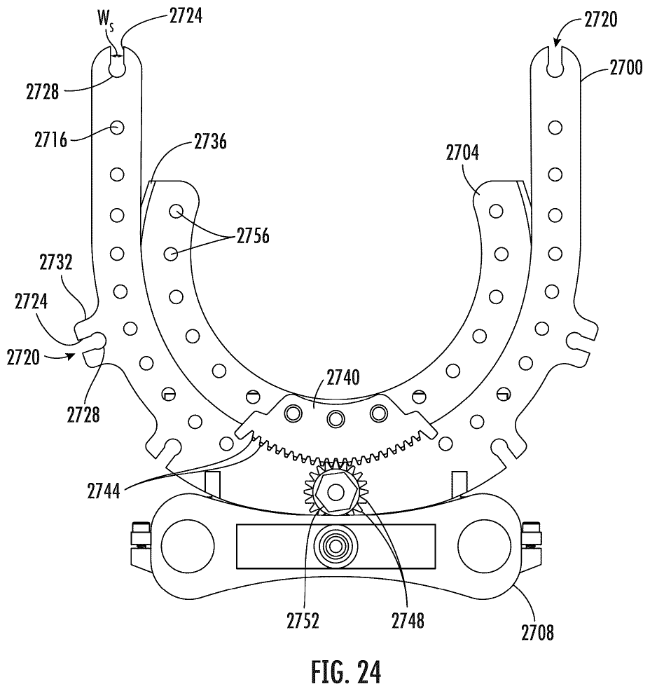

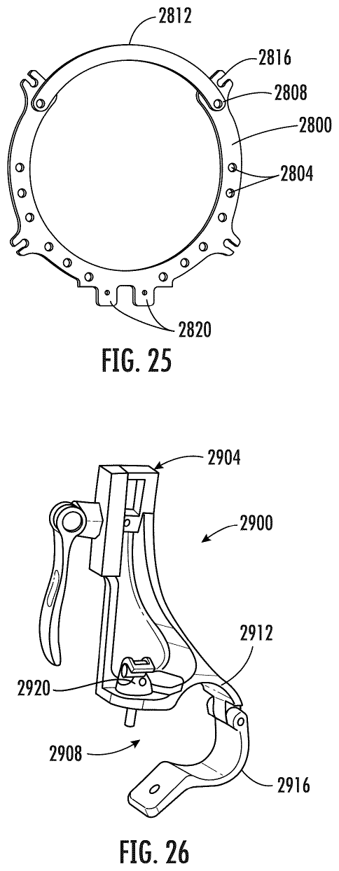

FIG. 24 illustrates the fixator member 2700, which includes a rotary portion 2704 configured to rotate relative to the fixator member 2700. The fixator member 2700 is configured to be coupled to a frame (not shown) via a carrier 2708. In the configuration illustrated in FIG. 24, the fixator member 2700 is not configured to rotate relative to the frame. For example, in the embodiment illustrated in FIG. 24, the rotary portion 2704 is configured to rotate about a longitudinal axis of the frame. The frame may be substantially similar to the frames 104, 404 and is not described in greater detail herein. The carrier 2708 is substantially similar to the carriers 110, 410 and is not described in greater detail herein.

The fixator member 2700 further includes a track 2712 configured to receive a portion of the rotary portion 2704 therein. In the configuration illustrated in FIG. 24, the track (similar to track 2516 shown in FIGS. 22-23) includes a groove. In other embodiments, the track may be another shape. In some embodiments, the track may extend along an entire inner circumference of the fixator member 2700. In other embodiments, the track may extend along one or more portions of the inner circumference of the fixator member 2700. The fixator member 2700 includes a plurality of holes 2716 configured to receive wires, pins, clamps, and/or fasteners therein. As shown in FIG. 24, the plurality of holes 2176 may be positioned about a circumference of the fixator member 2700.

In some embodiments, the fixator member 2700 includes a plurality of slots 2720 configured to receive a strut (such as the strut 2100 of FIG. 21). Each of the slots 2720 includes a substantially linear portion 2724 and a substantially round portion 2728. A width Wₛ of the substantially linear portion 2724 is narrower than a diameter of the substantially round portion 2728. The strut typically has a portion dimensioned to be slidably received within the substantially linear portion 2724 and rotatable within the substantially round portion 2728. A length of the portion of the strut that is slidably received in the substantially linear portion 2724 is wider than the width Wₛ and no larger than a diameter of the substantially round portion 2728, such that the portion of the strut can be rotated within the substantially round portion 2728 between a first configuration in which the strut can be inserted and/or removed from the slot 2720 and a second configuration in which the strut cannot be removed from the slot 2720.

The slots 2720 may be positioned at the ends of the fixator member 2700 and/or along a circumference of the fixator member 2700. In some embodiments, the slots 2720 may be formed in protrusions or cars 2732.

The rotary portion 2704 incudes a protrusion 2736 configured to be positioned within the track 2712. In some embodiments, the protrusion 2736 may extend along an entire outer circumference of the rotary portion 2704. In other embodiments, the protrusion 2736 may extend along one or more portions of the outer circumference of the rotary portion 2704.

A gear portion 2740 including a plurality of teeth 2744 may be coupled to the rotary portion 2704. In some embodiments, the plurality of teeth 2744 may extend along an entire circumference of the of the rotary portion 2794. In other embodiments, the plurality of teeth 2744 may extend along one or more portions of the rotary portion 2504. The plurality of teeth 2744 are configured to mesh with a plurality of teeth 2748 of an actuator gear 2752 coupled to the fixator member 2700. The actuator gear 2752 can be actuated (e.g., rotated) to reposition the rotary portion 2704. In the embodiment illustrated in FIG. 24, the actuator gear 2752 is a pinion gear. In other embodiments, the actuator gear 2752 may have another configuration, such as a worm gear. In some embodiments, a tool or handle may be used to actuate the actuator gear 2752. In other embodiments, the actuator gear 2752 may be rotated by a motor. In other embodiments, the actuator gear 2536 may be rotated by a motor. In some embodiments, the rotary portion 2704 and the fixator member 2700 may not include gears and/or teeth. In such embodiments, the gears and/or teeth may be replaced by detent mechanisms, a friction fit, and so forth.

With continued reference to FIG. 24, the rotary portion 2704 includes a plurality of holes 2756 along a circumference of the rotary portion 2704. The holes 2756 are configured to receive wires, pins, and/or clamps configured to receive wires and/or pins therein.

FIGS. 22-24 both show the rotary portions 2504, 2704 positioned radially inward relative to the fixator members 2500, 2700. This configuration may facilitate rotation of the rotary portions 2504, 2705. However, in other embodiments, the rotary portions 2504, 2704 may overlap or be positioned radially outwards of the fixator member 2500, 2700.

FIG. 23 illustrates a fixator member 2800 according to some embodiments. In the illustrated embodiment, the fixator member 2800 has a generally circular shape (covering, for example, between 180 and 320 degrees, for example, about 270 degrees). The fixator member 2800 includes a first plurality of holes 2804 configured to receive wires, pins, clamps, and/or fasteners therein. The fixator member 2800 includes a second plurality of holes 2808 positioned at or proximate an open end of the fixator member 2800. The second holes 2808 are configured for securing a closure element 2812 to the fixator member 2800 (e.g., via fasteners, not shown). The closure element 2812 is configured to close the open end of the fixator member 2800. In some embodiments, the closure element 2812 includes holes configured to receive wires, pins, holders, and/or fasteners.

The fixator member 2800 further includes slots 2816 substantially similar to the slots 2720 (FIG. 24). The fixator member 2800 further includes optional attachment features 2820 configured for securing the fixator member 2800 to a carrier such as the carriers 110, 410. In FIG. 24, the attachment features 2820 are protrusions. In other embodiments, the attachment features may have different shapes.

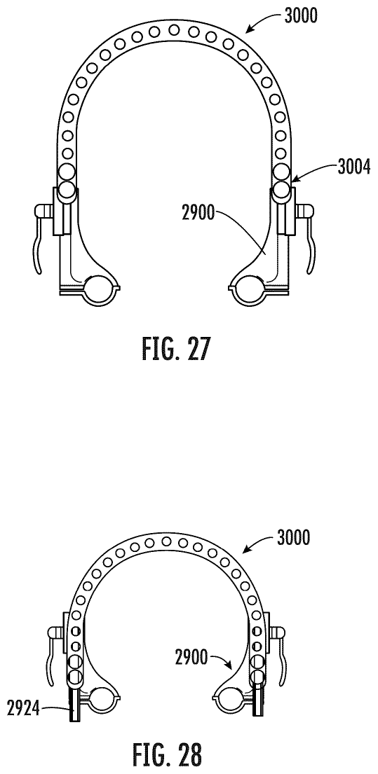

FIGS. 26-28 illustrate clamps 2900 that can be used to secure a fixator member 3000 to a frame, such as the frame 104, 404. In such embodiments, a carrier is not used.

The clamp 2900 includes a first clamp portion 2904 configured to engage an end 3004 of the fixator member 3000. The clamp 2900 includes a second clamp portion 2908 configured to releasably engage a frame, such as the frames 104, 404, of a fixation device, such as the fixation device 100, 400. For example, the second clamp portion 2909 may be configured to releasably engage elongate members, such as the elongate members 116, 416. In some embodiments, the second clamp portion 2908 may be shaped to conform to a shape of the elongate members of the frame. For example, in embodiments in which the elongate members are round tubular elements, the second clamp portion 2908 may include first and second arcuate portions 2912, 2916. In other embodiments, the clamp portion 2908 may be a different shape. In some embodiments, the first and second arcuate portions 2912, 2916 may be secured together via a fastener 2920. In some embodiments, the clamp 2900 may be substantially L-shaped, such that the first clamp portion 2904 is orthogonal to the second clamp portion 2908.

In some embodiments, clamps 2900 coupled to the ends of the fixator member 3000 are attached to the opposite elongate members of the frame (e.g., without using a carrier). In other embodiments, only one clamp 2900 may be coupled to the elongate members of the frame while the clamp 2900 remains unattached.

In some embodiments, as shown for example in FIG. 28, a height of the fixator member 3000 relative to the elongate members of the frame may be adjusted using linear extenders 2924 (e.g., which allow sliding along a rod and then locking) optionally configured at each of the open ends of the fixator member 3000. In some embodiments the extenders 2924 serve as guides and once the fixator member 3000 is pressed down along extenders 2923, the fixator member 3000 locks into place, for example, using a snap connection or other elasticity-based geometrical interference connection.

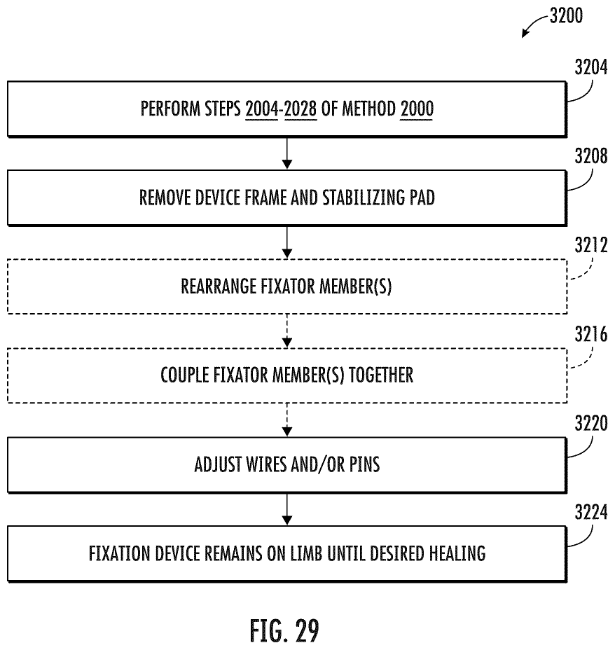

FIG. 29 is a flowchart of a method 3200 of using a fixation device such as the fixation devices 100, 400 both for immediate damage control and for long term treatment, according to some embodiments. When the fixation device 100, 400 is used for long term treatment, the fixation device 100, 400 remains deployed on the limb to serve as long term fixation, for example, for a time period of at least 1 month, at least 6 months, at least 1 year, or intermediate, longer or shorter time periods.

At 3204, the fixation device is deployed on the fractured limb for immediate damage control as described in steps 2004-2028 of method 2000. Optionally, the device is deployed soon after the fracture occurs or is identified, for example, within minutes from occurrence of the fracture, within hours, within a day or within a week.

At 3208, transforming the fixation device to serve for long term fixation includes removing the device frame 104, 404 and the stabilizing pad 112. This may leaving only the fixator members 108, 408 with the wires and/or pins held by them coupled to the patient. In some embodiments, the carriers 110, 410 connecting the fixator members 108, 408 to the frame 104, 404 are also removed.

At 3212, which is optional, the fixator members 108, 408 are re-arranged as needed.

At 3216, which is optional, two or more of the fixator members 108, 408 are then connected to each other, for example by struts. In some embodiments, such the fixator members 108, 408 may be coupled to each other via the struts before removing the frame 104, 404. Connecting the fixator members 108, 408 by struts may increase the device rigidity and strength, especially after the frame 104, 404 has been removed. A potential advantage of removing the frame 104, 404 may include providing for improved mobility of the patient. For example, if a leg fracture is being treated, removal of the frame 104, 404 may enable the patient to walk (with the fixator members still in place) instead of, for example, lying or sitting down. In some embodiments, additional fixator members 108, 408 may be coupled to the patient and are optionally connected to other fixator members 108, 408, for example by struts.

At 3220, the wires 448 and/or pins are adjusted for fine tuning alignment and stabilization of the fracture. Optionally, the position and/or orientation of the wires 448 may be adjusted. Optionally, an amount of tension applied by the wire 448 may be adjusted.

At 3224, the fixator members 108, 408 remain on the limb until desired healing of the fracture (1011). In some embodiments, imaging is performed (at any time point along the treatment period) to assess advancement of the healing process.

One possible use of the fixation device 100, 400 is as a damage control strategy, in which the fixation device 100, 400 is used as a transitional frame, stabilizing the limb until further treatment, such as surgery, can be provided. During surgery, the fixation device 100, 400 may be used intraoperatively to facilitate a more accurate reduction of the fractured long bone (e.g., to facilitate insertion of an intramedullary nail or a plate or an internally placed screw). After surgery, the fixation device 100, 400 may be left on the limb, e.g., to facilitate further healing of the fracture or it is removed.

In some embodiments of the disclosure, the frame 104, 404 may be removed and/or the components fixation device 100, 400 otherwise adjusted in preparation for a surgical correction, for example, insertion of a screw or a plate. Optionally, the surgery is performed while the limb is held using the fixation device 100, 400. In some cases, additional or alternative pins or wires 448 may be inserted for this surgical step (e.g., to clear access to a surgical region). In some cases, reconfiguration of the fixation device 100, 400 is used to help manipulate the limb during surgery. In some cases, manipulation of the limb is accomplished by adjustment of various elements of the fixation device 100, 400 as described herein (e.g., rotating or lengthening of parts of the fixation device 100, 400). In some embodiments, manipulation is manually, by removing rigidizing connections between parts of the fixation device 100, 400. In some cases adjustment of the fixation device 100, 400 is by using a hexapod adjustment method, on hexapod connections between fixator members 108, 408, for example, as described herein.

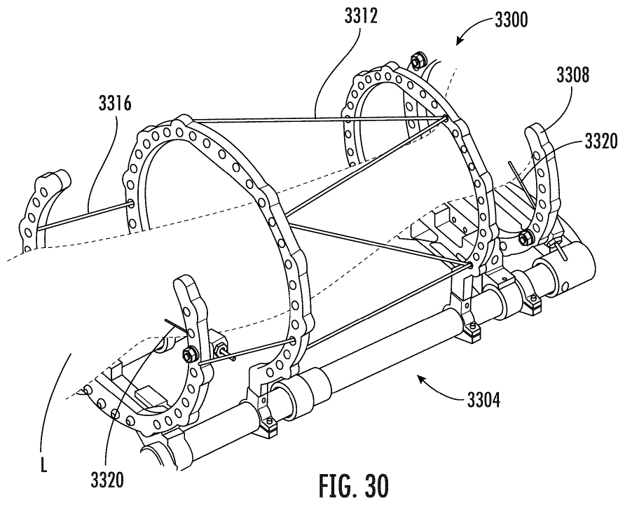

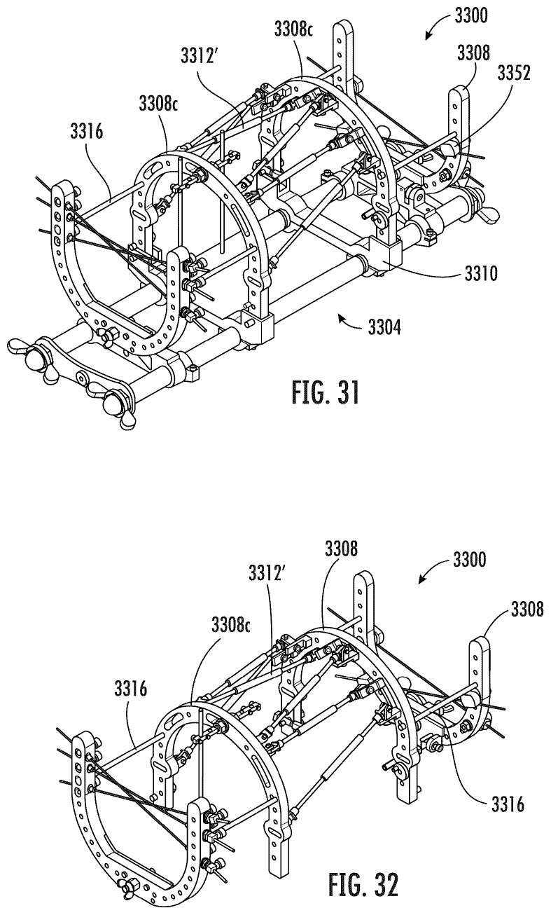

FIGS. 30-31 show examples of a long-term fixation device 3300 comprising a hexapod arrangement of struts, according to some embodiments.

In the example shown, the hexapod device 3300 comprises a frame 3304 to which fixator members 3308 are coupled. The frame 3304 may be substantially similar to the frames 104, 404 and is not described in greater detail herein. In some embodiments, the fixator members 3308 are substantially similar to the fixator members 108, 408, 1808, 2500, 2700, 2800, 3000, 3308. However, in some embodiments, for long term fixation, the frame 3304 may be removed, for example by releasing the attachment between the fixator members 3308 and the frame 3304. Such removal can improve the mobility of a patient, by reducing weight and/or geometry. Removal of the frame 3304 may also make it easier to cover the remaining portions of the fixation device 3300 coupled to the patient.

In some embodiments, two or more adjacent fixator members 3308 may be coupled to each other by six hexapod struts 3312 arranged in a hexapod configuration. A potential advantage of the hexapod configuration may include that three-dimensional fracture reduction may be achieved, allowing for gradual reconstruction of the bone. In some embodiments, one or more additional struts 3316 may be coupled between adjacent fixator members 3308 are arranged in a circular hexapod orientation, for example as shown. In such embodiments, the fixator members 3308 be secured to the limb L by one or more wires 3320, pins, and so forth.

In some embodiments, the hexapod struts 3312 and/or attachment features configured to couple the hexapod struts 3312 to the fixator members 3308 can be 3D printed, for example, based on an X-Ray or CT of the limb L, for example, before or during treatment of the fracture. In some embodiments of the disclosure, adjustable-length hexapod struts 3312′ (FIGS. 31-32) are provided. In some embodiments, these adjustable-length hexapod struts 3312′ may be motorized. In other embodiments, these adjustable-length hexapod struts 3312′ may be manually adjustable. In some embodiments of the disclosure, the 3D printed fixator member attachment features, which may be or include the actual attachment features coupling the struts 3312 to the fixator members 3308 or a stencil/overlay which indicates fixation locations for coupling the struts 3312 to the fixator members 3308, is provided in color and/or with instructional/guide markings. Optionally, the struts 3312 may be color coded as well or instead.

Subsequent to coupling the fixation device 3300 to the patient's limb, the frame 3304 can be removed to facilitate the surgical introduction of a permanent fixation device, for example an intramedullary nail, a plate or a screw. In some embodiments, the frame 3304 is used during such a surgical process.

In some embodiments, the hexapod struts 3312 may be coupled between the most proximal and most distal fixator members 3308. In other embodiments, such as the configuration shown in FIGS. 31-32, the hexapod struts 3312 may be coupled between intermediate fixator members 3308. In such embodiments, the hexapod struts 3312 may be coupled to the fractured limb via wires, pins, etc. Additionally or alternatively, in such embodiments, the intermediate fixator members 3308 may be coupled to adjacent fixator members 3308, such as the distal-most and/or proximal-most fixator members 3308, via additional struts 3316.

FIGS. 31-32 illustrate the fixation device 3300 including a hexapod arrangement of hexapod struts 3312′ extending between two intermediate fixator members 3308c. In some embodiments, when transferring the fixation device 3300 from an immediate use configuration (such as shown in FIG. 31) to a long term fixation configuration (such as shown in FIG. 32), the frame 3304 is removed. In some embodiments, the one or more carriers 3310 coupling the fixator members 3308 to the frame 3304 are also removed. The carriers 3310 are substantially similar to the carriers 110, 410 and are not described in detail herein. In some embodiments, the long-term fixation configuration of FIG. 32 is maintained stable due to the plurality of struts 3312, 2216 connecting the fixator members 3308 to each other. In some embodiments of the disclosure, the adjustable struts 3312′ are motorized. Optionally, the motors are controlled by a remote controller, for example, using a cellular connection.

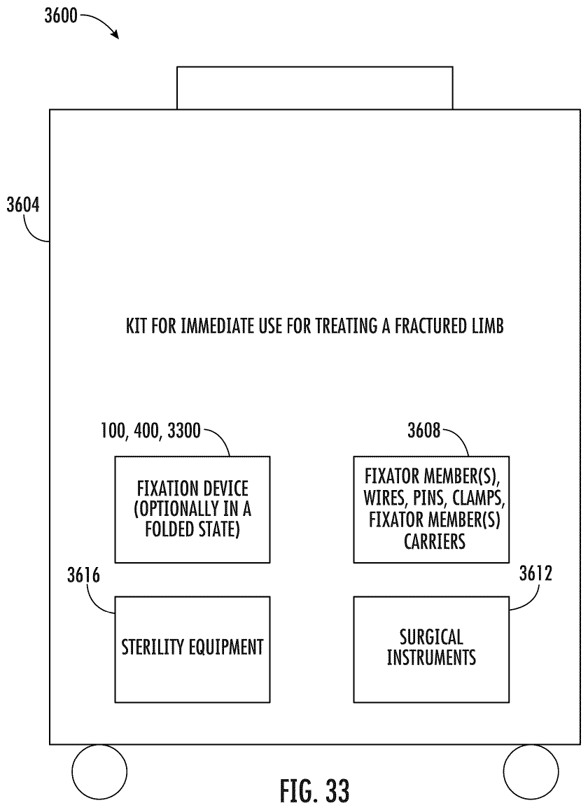

FIG. 33 illustrates a schematic diagram of a kit 3600 for immediate treatment of a fractured limb, according to some embodiments.

In some embodiments, the kit 3600 is provided in a package 3604, for example, in a bag, storage box, case and the like. In an example, the package comprises a case which can be converted into a platform or a table for use during device deployment. In some embodiments, the case can be rolled on the ground, for example via wheels.

In some embodiments, the kit 3600 includes a fixation device such as the fixation device 100, 400, 3300, optionally in a folded state; components 3608 usable with the fixation device, such as wires, pins, half-pins, clamps, fixator member carriers, fixator members, and the like. In some embodiments, the fixation device 100, 400, 3300 may be provided disassembled. In some embodiments, the fixation device 100, 400, 3800 may be provided assembled (e.g., fixator members and carriers may be coupled to the frame in either a folded or unfolded configuration).

Optionally, the kit 3600 may include surgical instruments 3612, for example a drill, a hammer, forceps and/or other tools. Optionally, the kit 3600 may include sterility equipment 3616, for example one or more drapes, gloves, sterilization materials, and the like. In some embodiments, one or more components of the kit are disposable and configured for single use only. Optionally, all kit components are disposable.

In some embodiments of the disclosure, the kit 3600 includes one or more stabilization pads, such as the stabilizing pads 112 (e.g., foam and/or expandable).

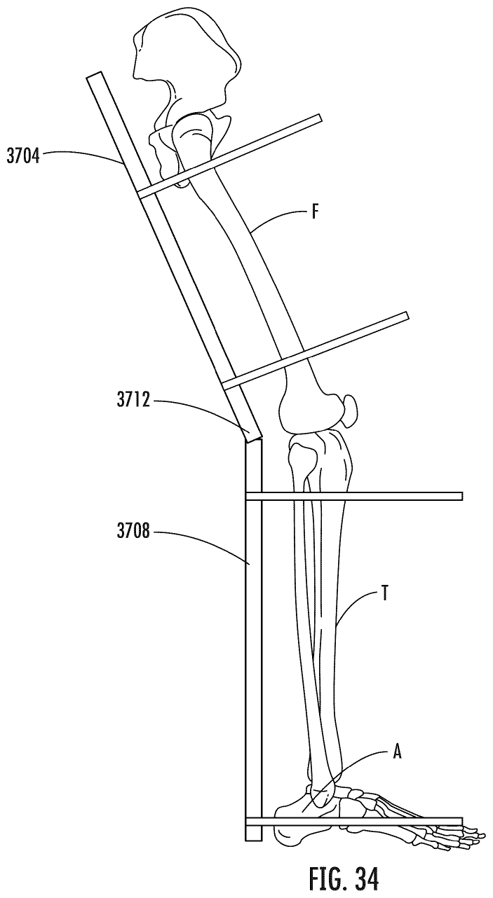

FIG. 34 shows a fixation device 300 having an offset in accordance with some embodiments of the disclosure.

A first fixation device section 3704 is attached to a femur F. A second fixation device section 3708 is attached to a tibia T and ankle A. The first and second fixation device sections may be substantially similar to the fixation devices 100, 400, 3300 and are not described in detail herein.

In some embodiments of the disclosure, the two fixation device sections 3704, 3708 are separate fixation devices which may be attached by a coupling 3712. In some embodiments, the coupling 3712 may define a fixed angle. Alternatively, the coupling 3712 may be adjustable.

In some embodiments of the disclosure, the two fixation device sections 3704, 3708 are part of one fixation device 3700, which includes a lockable hinge at coupling 3712 location.

It is noted that it may be desirable to set the angle of one part of the body relative to another when using multiple fixation devices 3704, 3708 to treat a single patient. FIG. 34 shows an example of how multiple fixation devices 3704, 3708 may be done using devices as described herein, for the leg. A similar arrangement may be used for the arm and/or for a limb relative to the trunk (e.g., pelvis fixation or shoulder fixation of the fixation device).

It is noted that one or both of the fixation device sections 3704, 3708 of FIG. 34 can include lengthening mechanisms (e.g., of the base and/or changing spacing of the fixation fixator members, potentially after the limb is attached thereto) and/or include rotation (e.g., using a ring within ring design or other designs, for example, as described herein).

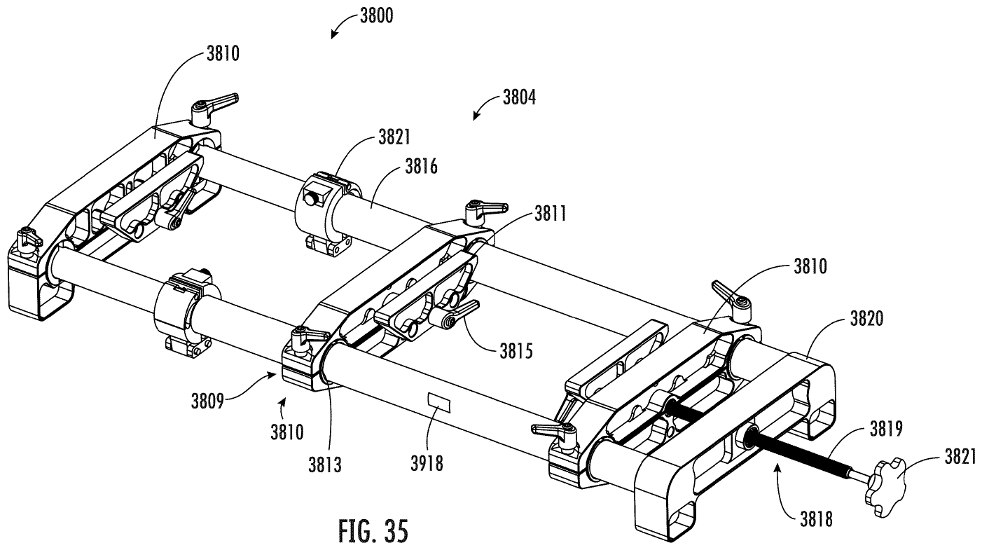

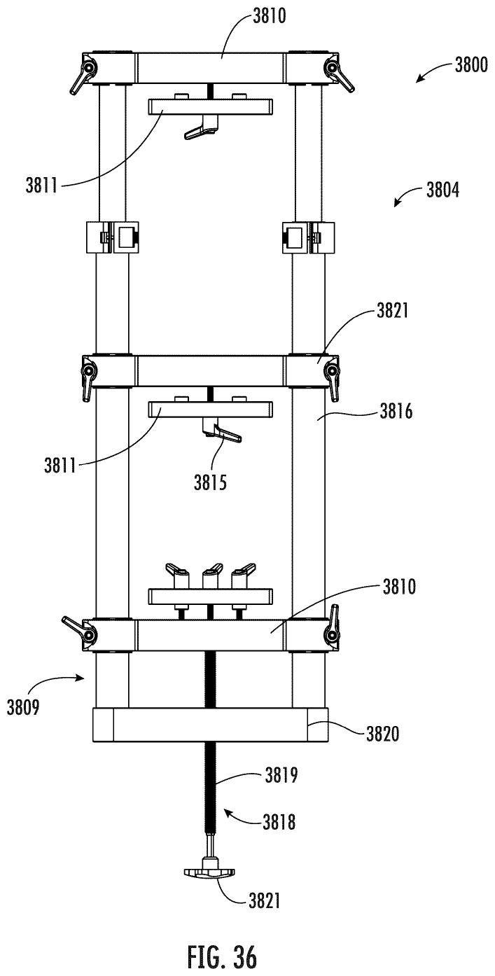

FIGS. 35-40 illustrate a fixation device 3800 configured for treatment of a fractured limb according to an embodiment of the present disclosure. The fixation device 3800 is substantially similar to the fixation devices 100, 400, 3300, and like parts will be referred to using like numbering.

The fixation device 3800 includes a frame 3804 including a pair of elongate members 3816 that are coupled together by at least one transverse connector 3820. In some embodiments, a transverse connector 3820 may be coupled to both ends of the elongate members 3816. In other embodiments, one or more of the carriers 3810 may be used in place of one or both of the traverse connector 3820. The ends of the elongate members 3816 do not rotate relative to the at least one transverse connector 3820. This allows the frame 3804 to provide rigid support to the injured limb as the injured limb is being secured within the fixation device 3800. Further, this rigidity can allow the frame 3804 to form a base of operations for preparing the fixation device 3800 (e.g., while positioning carriers 3810 along the frame 3804 and/or securing fixator members 3808 to the frame 3804), and when coupling the fixation device 3800 to the patient. This support can allow a healthcare practitioner to secure the fixation device 3800 to the patient without assistance.

In some embodiments, the elongate members 3816 may include rods, shafts, tubes, and so forth. In some embodiments, the elongate members 3816 include telescoping elements such that a length of the frame 3804 can be adjusted to conform to a length of the limb being treated by changing a length of the elongate members 3816. In such embodiments, the elongate members 3816 may include a locking feature 3817 configured to maintain the elongate members 3816 at a desired length. In other embodiments, a length of the elongate members 3816 may not be adjustable. Optionally, in embodiments in which the length of the frame 3804 is adjustable, the elongate members 3816 may include markings to assist with sizing the length of the frame 3804 based on the size of the fractured limb.

In some embodiments, the frame 3804 can be adjusted such that a length of the frame 3804 is substantially similar to a length of the fractured limb. For example, for a fracture in the tibia and/or fibula, the frame 3804 can be adjusted such that the length of the frame 3804 is substantially similar to a length of the patient's tibia and/or fibula. In some embodiments, a length of the frame 104 can extend from the patient's ankle to the patient's pelvis.

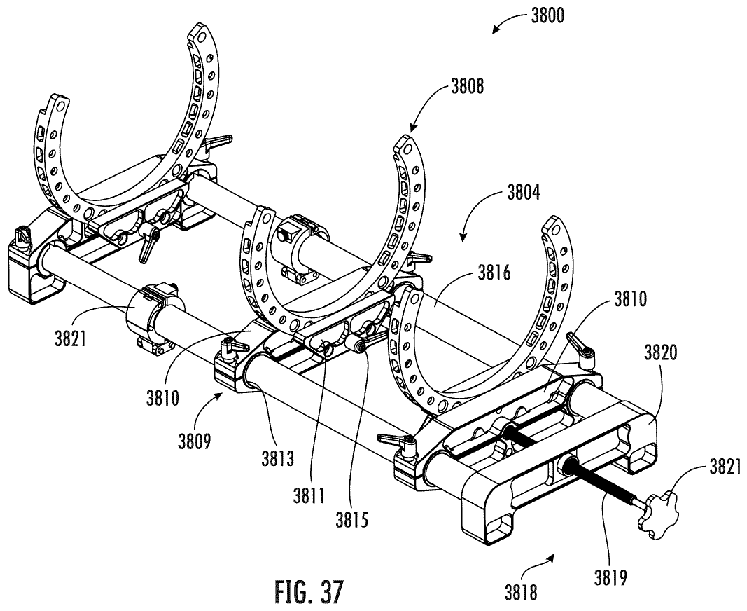

The device 3800 further includes a plurality of carriers 3810 that are coupled to the frame 3804 (e.g., via the elongate members 3816). The carriers 3810 include a fixator member clamp 3811 configured to engage a fixator member 3808 (FIG. 37). In some embodiments, the fixator member clamp 3811 has a vise-like configuration and is configured to tightly secure the fixator member 3808. In some embodiments, the fixator member clamp 3811 may include securing features such as teeth, protrusions, and so forth to assist with securing the fixator member 3808 within the fixator member clamp 3811. The fixator member clamp 3811 may include a locking feature 3815 to secure the fixator member clamp 3811 to the fixator member 3808.

The carriers 3810 are slidably coupled to the frame 3804 via clamping portions 3809. The clamping portions 3809 are configured to open such that the elongate members 3816 can be inserted into recesses 3813 of the clamping portions 3809. The clamping portions 3809 have first configuration in which the carrier 3810 are slidable along the elongate members 3816. The clamping portions 3809 have a second configuration in which the carrier 3810 is fixedly coupled to the elongate members 3816.

In some embodiments, a length of the carrier 3810 may be adjustable to change a distance between the elongate members 3816 of the frame 3804. In other embodiments, carriers 3810 may be provided in a variety of lengths such that the width of the frame 3804 (e.g., distance between the elongate members 3816) can be determined based on a size of the limb being treated. In such embodiments, a length of the transverse connector 3820 may also be adjustable and/or transverse connectors 3820 of varying lengths may be provided.

In some embodiments, the frame 3804 may be provided with one or more carriers 3810 attached. For example, in some embodiments, carriers 3810 may be provided proximate distal and proximal ends of the frame 3804. Additional carriers

In some embodiments, the frame 3804 further incudes a longitudinal adjuster 3818 configured to reposition one of the carriers 3810. In the illustrated configuration, the longitudinal adjuster 3818 includes an element 3819 that has first end that is coupled to the carrier 3810 and a second end that is coupled to a knob 3821. The element 3819 is threadably coupled to the transverse connector 3820. The element 3819 can be actuated, for example by the knob 3821, to reposition the carrier 3810 along a longitudinal extent of the frame 3804. In other embodiments, the element 3819 can be actuated via a motor. Actuation of the longitudinal adjuster 3818 can be used to assist with aligning bone fragments after the fixation device 3800. Actuation of the longitudinal adjuster 3818 can also fine tune the position of the carrier 3810 coupled to the longitudinal adjuster 3818. Although the embodiment illustrated in FIG. 35 includes one longitudinal adjuster 3818, in other embodiments, both ends of the frame 3804 may include longitudinal adjusters 3818. In some embodiments, the elongate members 3816 may include markings to assist with appropriate positioning of the carrier 3810 based on the fracture location, the ends of the injured limb, and so forth.

FIG. 37 illustrates a configuration in which the fixator members 3808 are coupled to the frame 3804. The frame 3804 provides a rigid base to which the fixator members 3808 can coupled and positioned as necessary to treat the fracture before the limb is brought to the frame 3804. This can allow a single practitioner to treat a fracture without assistance. For example, instead of arranging fixator members against the operating table and having an assistant hold the fixator members in position as they are being arranged and coupled to the patient, the frame 3804 can hold the fixator members 3808 in position as the fixator members 3808 are being coupled to the patient. Further, once fixator members 3808 have been secured to distal and proximal ends of the fractured limb, the longitudinal adjuster 3818 can be actuated to align bone fragments.

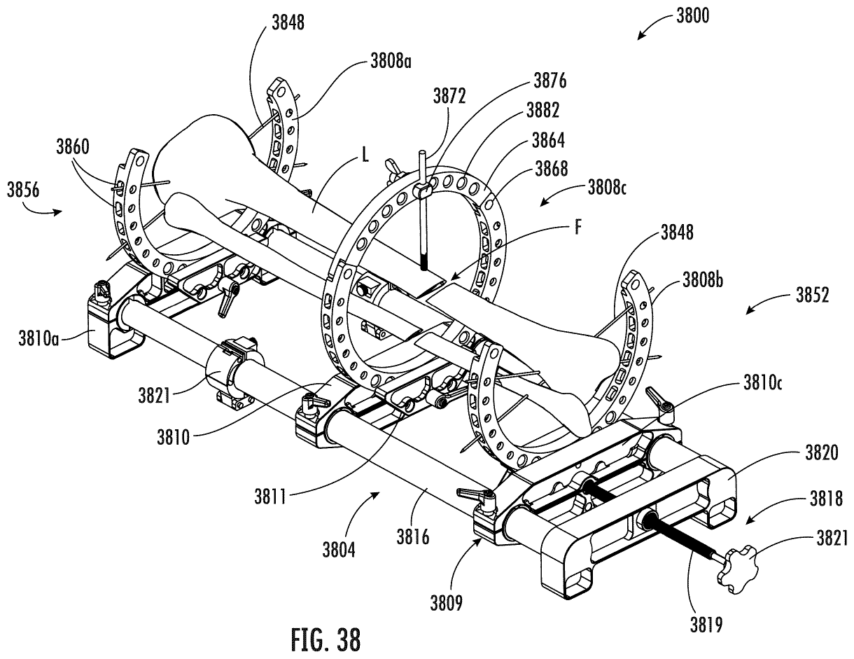

FIG. 38 illustrates a perspective view of the fixation device 3800 coupled to a fracture F in a limb L. As shown in FIG. 38, the elongate members 3816 have been adjusted such that the frame 3804 spans a length of limb L. The proximal carriage 3810a has been positioned near a proximal end 3856 of the frame 3804 such that the proximal fixator member 3808a is oriented to engage a proximal portion of the fractured limb L. The distal carriage 3810b has been positioned near a distal end 3852 of the frame 3804 such that the distal fixator member 3808b is oriented to engage a distal portion of the fractured limb L. An intermediate carriage 3810c has been positioned proximate a location of the fracture F. In some embodiments, positioning the carriages 3810a-3810c and the fixator members 3808a-3808c, the limb L may be brought to and positioned within the frame 3804.

As shown in FIG. 38, wires 3848 are inserted into the limb L via the holes 3860 in the distal and proximal fixator members 3808a, 3808b. At this point, optionally, imaging may be done to assess the fracture F. Further, if necessary, the longitudinal adjuster 3818 may be actuated to pull traction on the limb L, to align the bone fragments in an axial direction. Optionally, imaging may again may be used to determine the position of the bone fragments. In the configuration illustrated in FIG. 38, a fixator member closure element 3864 has been coupled to the intermediate fixator member 3808c, for example via the holes 3868. The closure element 3833 allows elements such as pins, wires, etc. to engage the limb from above. For example, as shown in FIG. 38, a pin 3872 is coupled to the fixator member closure element 3864 via a clamp 3876 coupled to a hole 3880 in the fixator member closure element 3864. In the configuration illustrated in FIG. 38, the pin 3872 is used to push a bone fragment in the downward direction. In other configurations, pins 3872 can be coupled to the fixator member 3808c to push the bone fragments in the lateral and/or medial direction.

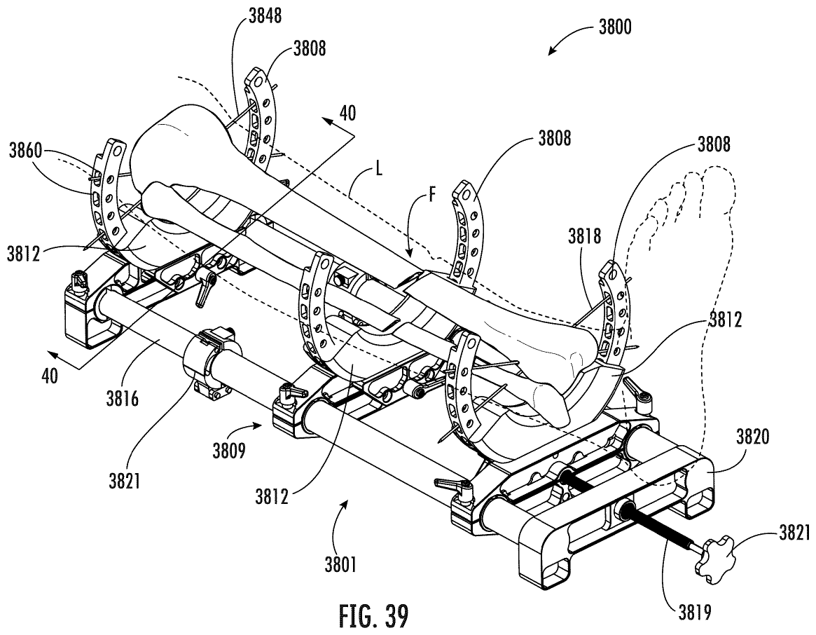

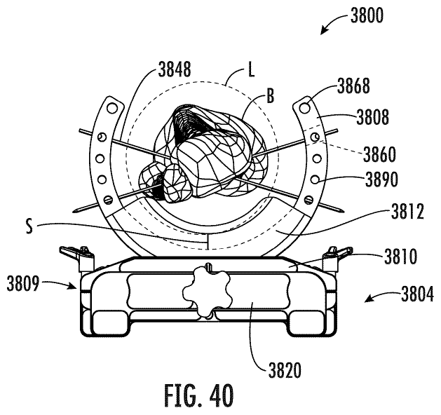

FIG. 39 illustrates another configuration in which the limb L is coupled to the fixation device 3800. The configuration of FIG. 39 is similar to the configuration of FIG. 38, except that the configuration of FIG. 39 does not include the fixator member closure element 3864 and the pin 3872 and the configuration of FIG. 39 includes stabilizing pads 3812. FIG. 40 illustrates a section view of FIG. 42 taken at lines 40-40.

In the configuration of FIG. 39, a stabilizing pad 3812 is coupled to each of the fixator members 3808. The stabilizing pads 3812 are configured to support the soft tissue of the limb L and prevent the fixator members 3808 from becoming pressure points on the limb L. For example, the stabilizing pads 3812 may be configured to center the limb L in the fixator members 3808. The stabilization provided by the stabilizing pads 3812 is such that an assistant is not required to hold the limb L in position as the fixation device 3800 is being coupled to the patient. In some embodiments, the stabilizing pads 3812 are sized to position the soft tissue of the limb L a predefined spacing S (FIG. 40) between the patient's skin and the fixator members 3808. Although FIGS. 39-40 show separate stabilizing pads 3812 coupled to each fixator member 3808, in other embodiments, the stabilizing pad may be a single pad that spans the distance between adjacent fixator members 3808. The stabilizing pad 3812 may have the same material properties and include the same materials as described with regard to the stabilizing pad 112.

In some embodiments, such as the embodiment shown in FIGS. 49-51, a stabilizing pad 3812′ may be directly coupled to one or more of the carriers 3810, for example via the fixator member clamp 3811. Although FIG. 49 shows both carriers 3810 coupled to the stabilizing pad 3812′ instead of a fixator member, in other aspects, some of the carriers 3810 coupled to the frame 3800 may be coupled to fixator members 3808, and other carriers 3810 may be coupled to the stabilizing pads 3812′.

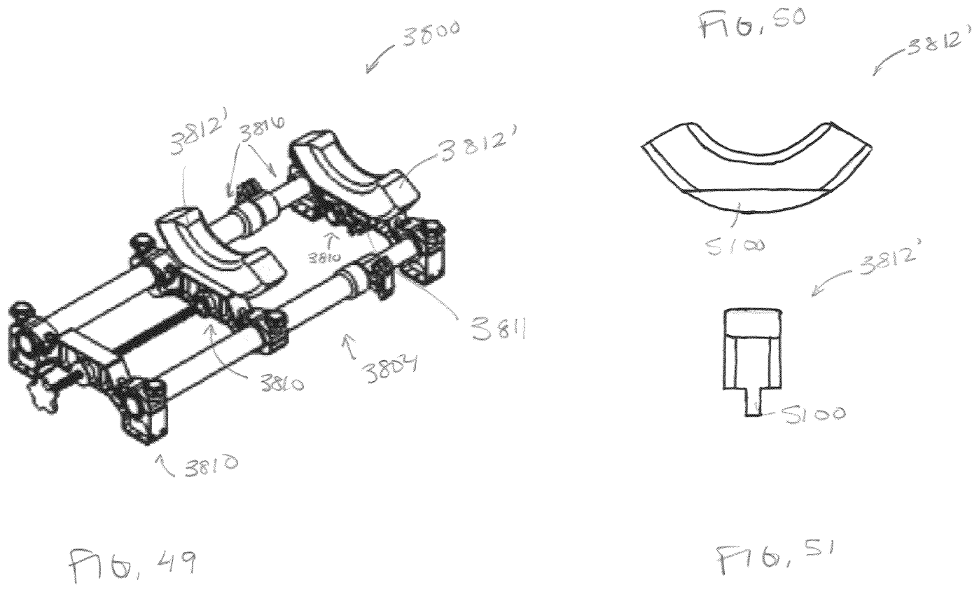

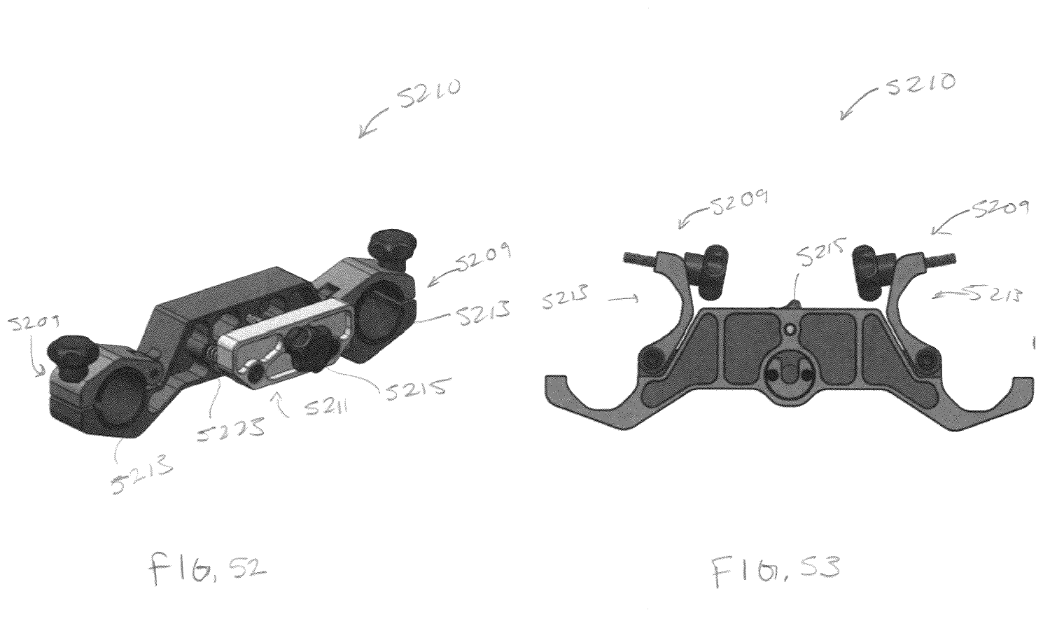

FIGS. 50-51 illustrate the stabilizing pads 3812′. The stabilizing pads 3812′ are substantially similar to the stabilizing pads 3812 and differ from the stabilizing pads 3812 only in that the stabilizing pads 3812′ include a protrusion 5100 configured to be engaged and/or grasped by the fixator clamp 3811.

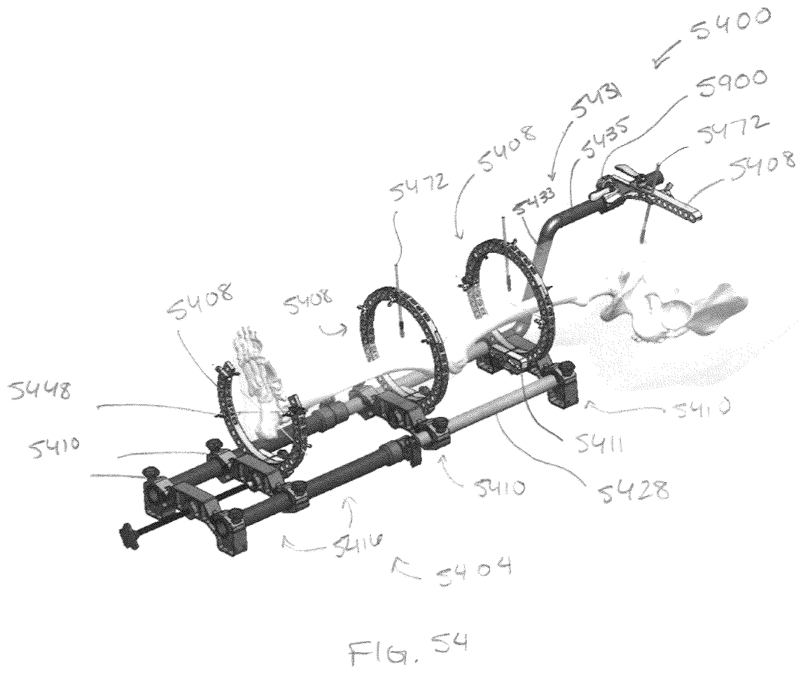

FIGS. 52-53 illustrate a carrier 5210 that can be used with the fixation device 3800 according to some embodiments. The carrier 5210 is substantially similar to the carrier 3810, and like parts between the carrier 5210 and the carrier 3810 are referred to using like numbers. The carriers 5210 include a fixator member clamp 5211 configured to engage a fixator member or a stabilizing pad (not shown). In some embodiments, the fixator member clamp 5211 has a vise-like configuration and is configured to tightly secure the fixator member and/or the stabilizing pad. In some embodiments, the fixator member clamp 5211 may include securing features such as teeth, protrusions, and so forth to assist with securing the fixator member and/or the stabilizing pad within the fixator member clamp 5211. The fixator member clamp 5211 may include a locking feature 5215 to secure the fixator member clamp 5211 to the fixator member and/or the stabilizing pad. In some embodiments, the carrier 5210 includes a spring 5223 configured to bias the fixator member clamp 5211 away from the carrier 5210. In such embodiments, the spring 5223 may push the fixator member clamp 5211 away from the carrier 5210 when the locking feature 5215 has been released, thereby facilitating the insertion and/or removal of fixator members and/or stabilizing pads into the fixator member clamp 5211.

The carriers 5210 are slidably coupled to the frame 3804 via clamping portions 5209. The clamping portions 5209 are configured to open such that the elongate members 3816 can be inserted into recesses 5213 of the clamping portions 5209. The clamping portions 5209 have first configuration in which the carrier 3810 are slidable along the elongate members 3816. The clamping portions 5209 have a second configuration in which the carrier 3810 is fixedly coupled to the elongate members 3816.

FIGS. 41-45 illustrate the fixator members 3808 and the fixator member closure element 3864 according to embodiments of the present disclosure. In the illustrated configuration, the fixator member 3808 has a partial ring shape. The fixator member closure element 3864 can be coupled to the fixator member 3808 to produce a full ring shape. In other embodiments, the fixator member 3808 have a different shape, for example, a U-shape, an arc shape, a ring shape, a partial ring shape, a bow shape, a horseshoe shape, a half-horseshoe shape, and/or a post shape.

The fixator member 3808 includes a first plurality of holes 3860 that extend through a thickness T of the fixator member 3808 (FIGS. 44-45). The first plurality of holes 3860 is configured to receive the wires 3848. In some embodiments, the first plurality of holes 3860 includes spacing features 3886 (inset 47A) configured to orient the wires 3848 coupled within the same fixator member 3808 in different planes. This prevents wires 3848 coupled to the same fixator member 3808 from interfering with each other. In the illustrated embodiment, the spacing feature 3886 includes holes 3887 positioned within a plate 3889 positioned within the opening 3860. In the illustrated embodiment, the spacing feature includes two holes 3887. In other embodiments, the spacing feature 3886 may include more or fewer holes 3887.

The fixator member 3808 includes a second plurality of holes 3890 formed in a width W of the fixator member 3808. The second holes 3890 are orthogonal to the first holes 3860. In some embodiments, the second holes 3890 may be configured to receive fasteners (not shown) configured to secure wires 3848 inserted through the holes 3860 to the fixator member 3808. In other embodiments, the second holes 3890 may be configured to receive clamps and/or holders (e.g., the holders 452) configured to receive the wires 3848. In the illustrated embodiment, the holes 3890 include indica 3894. The indicia 3894 on a particular opening 3890 illustrate acceptable orientations for wires 3848 visible through (e.g., when the wires 3848 are inserted into the holes 3860) or coupled to a particular opening 3890. In other embodiments, the indicia 3894 can have other configurations, for example, matching colors, letters, numbers, and so forth may be used to indicate pairs of holes 3890 through which a particular wire 3848 may be inserted (or visible through). In some embodiments, the holes 3890 may include second indica 3898 indicating acceptable orientations for a pin 3872 coupled to a particular opening 3890.

The fixator member 3808 includes a third plurality of holes 3868 proximate the ends 3902 of the fixator member 3808. The holes 3868 are configured for coupling the fixator member closure element 3864 to the fixator member 3808. In some embodiments, the ends 3902 of the fixator member 3808 may include stepped portions 3906 (FIG. 44).

The fixator member closure element 3864 include a first plurality of holes 3882 configured to receive a pin 3872 therethrough. In some embodiments, the holes 3882 may include indica 3910 indicating acceptable orientations for a pin 3872 coupled to a particular opening 3890.

The fixator member closure element 3864 includes a second plurality of holes 3868′ proximate the ends 3914 of the fixator member closure element 3864. The holes 3868′ are configured for coupling the fixator member closure element 3864 to the fixator member 3808. For example, the holes 3868, 3868′ can be aligned with each other and a fastener (not shown) can be inserted therethrough. In some embodiments, the ends 3914 of the fixator member closure element 3864 may include stepped portions 3920 (FIG. 44).

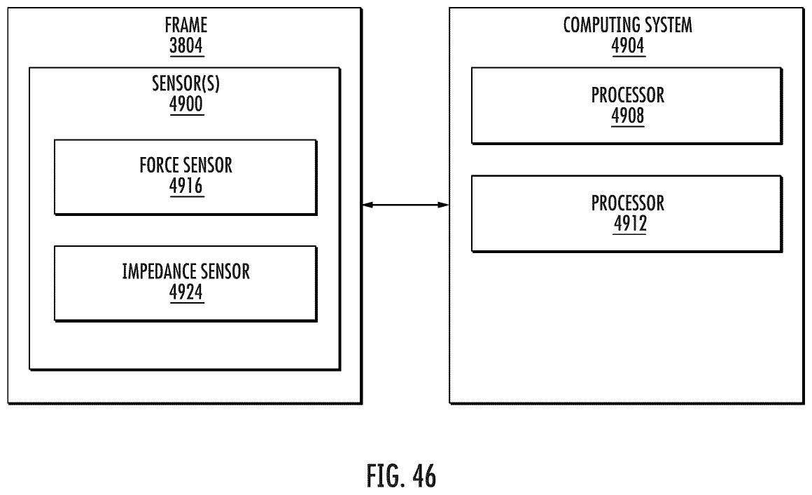

As shown in FIG. 46, In some embodiments, the frame 3804 may include one or more sensors 4900 (FIGS. 4647 and 35). In some embodiments, the one or more sensors 4900 may be coupled to the elongate members 3816. The sensor(s) 4900 may be configured to determine information indicative of the healing status of the fracture F. The sensor(s) 4900 are configured to communicate with a computing system 4904 including a processor 4908 and a memory 4912.

For example, in some embodiments, the sensor(s) 4900 may include one or more force sensors 4916 configured to determine the forces within the frame 3804. As fractures heal, the bone fragments can no longer move relative to each other, which causes forces in the fixation device 3800 to increase. In such embodiments, the computing system 4904 is configured to receive force data determined by the force sensor(s) 4916. Example force sensors 4916 may include load cells, strain gages, force sensing resistors, and so forth. The computing system 4904 may be configured to determine healing progress based on the determined force information. For example, in some embodiments, the memory 4912 may include a force database 4920 including a lookup table that correlates healing progress to sensed forces in the frame 3804 with fracture healing data. In such embodiments, the computing system 4904 may compare the determined force data to the data in the lookup table to determine healing progress of the fracture. In other embodiments, the computing system 4904 may compare the determined force information to a threshold. In response to the determined force information being greater than or equal to the threshold, the computing system 4094 may determine that the fracture has healed.

In some embodiments, the sensor(s) 4900 may include one or more impedance sensor(s) 4924 configured to determine an impedance along the length of the bone.

In some embodiments, the sensor(s) 4900 may include one or more sensors configured to determine the patient's vital signs. In such embodiments, the sensor(s) may include temperature sensors, heart rate sensors, and so forth. In other aspects, the frame 3804 may include the computing system 4904, and the computing system 4904 may be configured to receive information indicative of the patient's vital signs from sensor(s) coupled to the patient. In some embodiments, these sensor(s) may be coupled to the frame. In other embodiments, these sensor(s) may be coupled to the patient and configured to wirelessly transmit information indicative of the patient's vital signs to the system 4904.

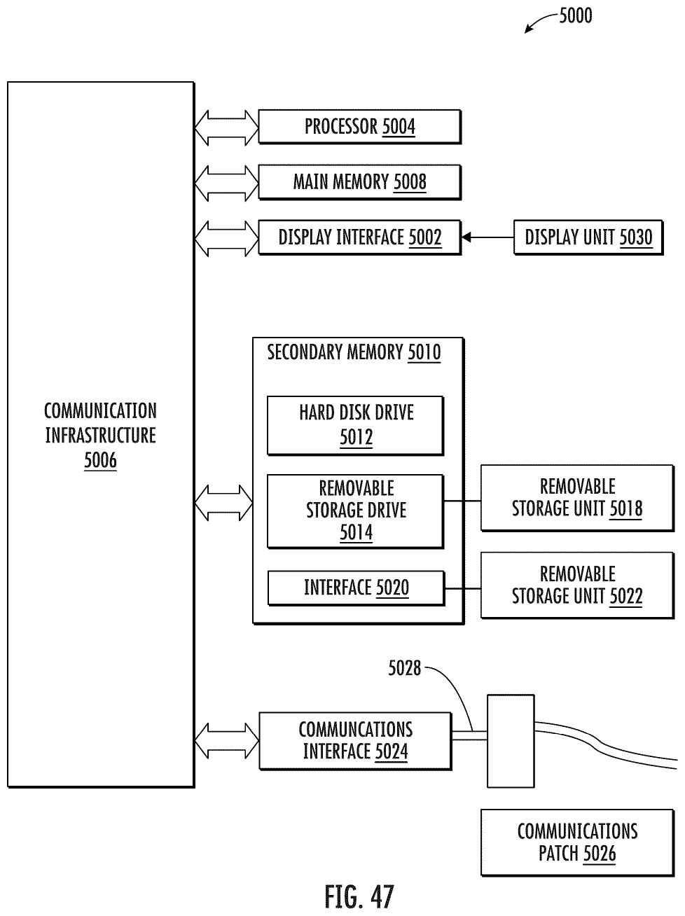

FIG. 47 presents an example system diagram of various hardware components and other features, for use in accordance with an embodiment of the present disclosure. Embodiments of the present disclosure may be implemented using hardware, software, or a combination thereof and may be implemented in one or more computer systems or other processing systems. In one example variation, embodiments described herein may be directed toward one or more computer systems capable of carrying out the functionality described herein of the fixation device 3800. An example of such a computer system 5000 is shown in FIG. 47.

The computer system 5000 includes one or more processors, such as processor 5004. The processor 5004 is connected to a communication infrastructure 906 (e.g., a communications bus, cross-over bar, or network). The processor 5004 may include a processor for the local computing system 4904 of FIG. 46. Various software embodiments are described in terms of this example computer system. After reading this description, it will become apparent to a person skilled in the relevant art(s) how to implement embodiments described herein using other computer systems and/or architectures.

Computer system 5000 may include a display interface 5002 that forwards graphics, text, and other data from the communication infrastructure 5006 (or from a frame buffer not shown) for display on a display unit 5030. Computer system 5000 also includes a main memory 5008, preferably random access memory (RAM), and may also include a secondary memory 5010. The secondary memory 5010 may include, for example, a hard disk drive 5012 and/or a removable storage drive 5014, representing a floppy disk drive, a magnetic tape drive, an optical disk drive, etc. The removable storage drive 5014 reads from and/or writes to a removable storage unit 5018 in a well-known manner. Removable storage unit 5018, represents a floppy disk, magnetic tape, optical disk, etc., which is read by and written to removable storage drive 5014. As will be appreciated, the removable storage unit 5018 includes a computer usable storage medium having stored therein computer software and/or data.

In alternative embodiments, secondary memory 5010 may include other similar devices for allowing computer programs or other instructions to be loaded into computer system 5000. Such devices may include, for example, a removable storage unit 5022 and an interface 5020. Examples of such may include a program cartridge and cartridge interface (such as that found in video game devices), a removable memory chip (such as an erasable programmable read only memory (EPROM), or programmable read only memory (PROM)) and associated socket, and other removable storage units 5022 and interfaces 5020, which allow software and data to be transferred from the removable storage unit 5022 to computer system 5000. In an example, memory for the computing system 4904 may include the main memory 5008, the secondary memory 5010, the removable storage drive 5014, the removable storage unit 5018, the removable storage unit 5022, etc.

The computer system 5000 may also include a communications interface 5024. Communications interface 5024 allows software and data to be transferred between computer system 5000 and external devices. Examples of communications interface 5024 may include a modem, a network interface (such as an Ethernet card), a communications port, a Personal Computer Memory Card International Association (PCMCIA) slot and card, etc. Software and data transferred via communications interface 5024 are in the form of signals 5028, which may be electronic, electromagnetic, optical or other signals capable of being received by communications interface 5024. These signals 5028 are provided to communications interface 5024 via a communications path (e.g., channel) 5026. This path 5026 carries signals 5028 and may be implemented using wire or cable, fiber optics, a telephone line, a cellular link, a radio frequency (RF) link and/or other communications channels. In this document, the terms “computer program medium” and “computer usable medium” are used to refer generally to media such as a removable storage drive, a hard disk installed in a hard disk drive, and/or signals 5028. These computer program products provide software to the computer system 5000. Embodiments described herein may be directed to such computer program products.

Computer programs (also referred to as computer control logic) are stored in main memory 5008 and/or secondary memory 5010. Computer programs may also be received via communications interface 5024. Such computer programs, when executed, enable the computer system 5000 to perform various features in accordance with embodiments described herein. In particular, the computer programs, when executed, enable the processor 5004 to perform such features. Accordingly, such computer programs represent controllers of the computer system 5000.

In variations where embodiments described herein are implemented using software, the software may be stored in a computer program product and loaded into computer system 5000 using removable storage drive 5014, hard disk drive 5012, or communications interface 5020. The control logic (software), when executed by the processor 5004, causes the processor 5004 to perform the functions in accordance with embodiments described herein as described herein. In another variation, embodiments are implemented primarily in hardware using, for example, hardware components, such as application specific integrated circuits (ASICs). Implementation of the hardware state machine so as to perform the functions described herein will be apparent to persons skilled in the relevant art(s).

In yet another example variation, embodiments described herein are implemented using a combination of both hardware and software.

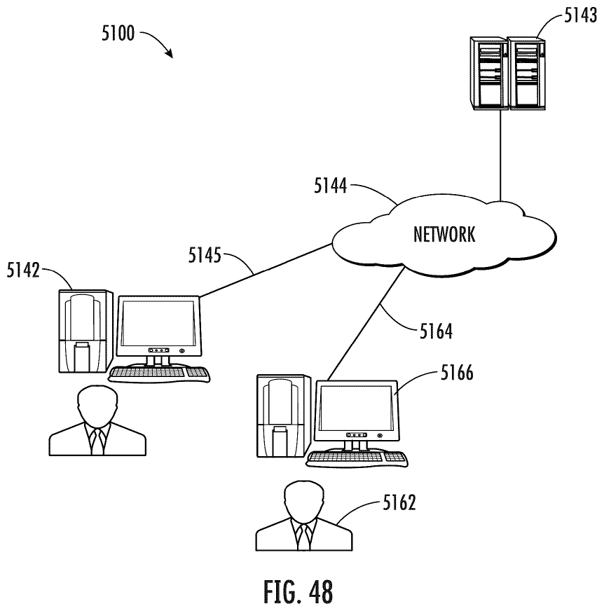

FIG. 48 is a block diagram of various example system components for use in accordance with embodiments of the present disclosure. FIG. 48 shows a communication system 5100 usable in accordance with embodiments described herein. The communication system 5100 may include one or more users 5160, 5162 and one or more terminals 5142, 5166. For example, terminals 5142, 5166 may include the control system 5120 or a related system, and/or the like. In one embodiment, data for use in accordance with embodiments described herein is, for example, input and/or accessed by users 5160, 5162 via terminals 5142, 5166, such as personal computers (PCs), minicomputers, mainframe computers, microcomputers, telephonic devices, or wireless devices, such as personal digital assistants (“PDAs”) or a hand-held wireless devices coupled to a server 5143, such as a PC, minicomputer, mainframe computer, microcomputer, or other device having a processor and a repository for data and/or connection to a repository for data, via, for example, a network 5144, such as the Internet or an intranet, and couplings 5145, 5146, 5164. The couplings 5145, 5146, 5164 include, for example, wired, wireless, or fiberoptic links. In another example variation, the method and system in accordance with embodiments described herein operate in a stand-alone environment, such as on a single terminal.

FIG. 54 illustrates a fixation device 5400 configured to treat a pelvis of the patient. The fixation device 5400 is substantially similar to the fixation devices 100, 400, and 3800, and is only described in detail to the extent to which it differs from the fixation devices 100, 400, 3800. Like numbering is used to indicate like parts between the fixation device 5400 and the fixation devices 100, 400, 3800.

In the embodiment illustrated in FIG. 54, the frame 5404 is in an extended position, such that the frame 5404 extends between the patient's ankle and the patient's pelvis. The elongate members 5416 and the extension 5428 are in an extended configure such that the frame 5404 extends from the patient's ankle to the patient's femur. The frame 5404 further includes a pelvis adaptor 5431. The pelvis adaptor 5431 includes a first portion 5433 and a second portion 5435. In some aspects, the first portion 5433 and the second portion 5435 are substantially orthogonal to each other. The first portion 5433 is configured span a distance between the frame 5404 and the top of the patient's thigh. The second portion 5435 is configured to overlie an upper portion of the patient's femur and overlie the patient's pelvis. In some aspects, a length of the first portion 5433 and/or the second portion 5435 can be adjusted.

Fixator members 5408 are coupled to the frame 5404 via carriers 5210 or clamps 5900. The clamps 5900 are substantially similar to the clamps 2900 (FIG. 26). The fixator members 5408 are coupled to the patient's limb via wires 5448 or pins 5472. One or more struts (not shown) may be positioned between adjacent fixator members 5408 to help stabilize the limb.

The foregoing detailed description of various aspects, features and embodiments of the invention includes, out of necessity, a detailed description of specific exemplary components. It will be apparent to one of ordinary skill in the art, upon reading the within description that these components may be interchangeable, and moreover, components of one embodiment may be added to another embodiment to obtain the same feature, form or function.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present disclosure. To the extent that section headings are used, they should not be construed as necessarily limiting. In addition, any priority document(s) of this application is/are hereby incorporated herein by reference in its/their entirety.

Clause 1. A fixation device configured to stabilize a fracture in a long bone, the fixation device comprising: a frame including an elongate member; a first carrier configured to be coupled to a first fixator member and configured to be coupled to the elongate member; and a second carrier configured to be coupled to a second fixator member and configured to be coupled to the elongate member.

Clause 2. The fixation device of clause 1, wherein the first carrier has a first configuration in which the first carrier is slidable along the elongate member and a second configuration in which the first carrier is fixedly coupled to the elongate member.

Clause 3. The fixation device of clause 1, wherein the elongate member includes telescoping elements.

Clause 4. The fixation device of clause 1, wherein the first fixator member and the second fixator member are is configured to be coupled to the fracture.

Clause 5. The fixation device of clause 4, wherein the frame is removed after the first fixator member and the second fixator member have been coupled to the fracture.

Clause 6. The fixation device of clause 1, wherein the elongate member is a first elongate member, and wherein the frame includes a second elongate member, and wherein the first carrier includes a first attachment feature configured to engage the first elongate member and a second attachment feature configured to engage the second elongate member, and wherein the first and second attachment features are not rotatable relative to the first and second elongate members.

Clause 7. The fixation device of clause 1, further comprising: a force sensor coupled to the frame, the force sensor configured to sense forces applied to the frame by the fracture; and a computing system configured to receive forces sensed by the force sensor and determine, based on the received sensed forces, a healing state of the fracture.

Clause 8. A method of treating a facture on a long bone having a first end and a second end, the method comprising: positioning a first carrier along an elongate member of a frame of a fixation device such that the first carrier is oriented to be proximate the first end of the long bone when the frame is coupled is coupled to the long bone; positioning a second carrier along the elongate member of the frame such that the second carrier is oriented to be proximate the second end of the long bone when the frame is coupled is coupled to the long bone; coupling a first fixator member to the first carrier; coupling a second fixator member to the second carrier; and coupling the first fixator member and the second fixator member to the fracture.

Clause 9. The method of clause 8, wherein the fixation device is coupled to the fracture after the first and second carriers have been coupled to the elongate member and after the first and second fixator members have been coupled to the first and second carriers.

Clause 10. The method of clause 8, wherein the first carrier has a first configuration in which the first carrier is slidable along the elongate member and a second configuration in which the first carrier is fixedly coupled to the elongate member.

Clause 11. The method of clause 8, wherein the method includes adjusting a length of the elongate member using telescoping elements in the elongate member.

Clause 12. The method of clause 8, further comprising removing the frame after the first fixator member has been coupled to the fracture.

Clause 13. The method of clause 8, wherein the elongate member is a first elongate member, wherein the frame includes a second elongate member, and wherein the first carrier includes a first attachment feature configured to engage the first elongate member and a second attachment feature configured to engage the second elongate member, and wherein the first and second attachment features are not rotatable relative to the first and second elongate members.

Clause 14. The method of clause 8, further comprising manipulating bone fragments of the fracture to reduce the fracture.

Clause 15. The method of clause 14, wherein manipulating the bone fragments includes repositioning at least one of the first fixator member and the second fixator member by a longitudinal adjuster coupled to the frame.

Clause 16. The method of clause 14, wherein manipulating the bone fragments includes rotating the first fixator member relative to the first carrier.

Clause 17. The method of clause 14, wherein manipulating the bone fragments includes inserting a pin into a bone fragment of the fracture to reposition the bone fragment, the pin coupled to a third fixator member coupled to the elongate member via a third carrier.

Clause 18. A portable fixation table configured to stabilize a fracture in a long bone, the portable fixation table comprising: a frame including an elongate member; a first carrier configured to be coupled to a first fixator member and is configured to be coupled to the elongate member; and a second carrier configured to be coupled to a second fixator member and is configured to be coupled to the elongate member; wherein the frame is configured to be moveable between a collapsed state in which the first and second fixator members lie in a plane that is generally parallel to a plane in which the elongate member lies, and a deployed state in which the first fixator member lies in a plane that is generally perpendicular to the plane in which the elongate member lie and in which the second fixator member lies in a plane that is generally perpendicular to the plane in which the elongate member lies.

What is claimed is:

1. A fixation device configured to stabilize a fracture in a long bone, the fixation device comprising:
a frame including an elongate member;
a first carrier configured to be coupled to a first fixator member and configured to be coupled to the elongate member; and
a second carrier configured to be coupled to a second fixator member and configured to be coupled to the elongate member.

2. The fixation device of claim 1, wherein the first carrier has a first configuration in which the first carrier is slidable along the elongate member and a second configuration in which the first carrier is fixedly coupled to the elongate member.

3. The fixation device of claim 1, wherein the elongate member includes telescoping elements.

4. The fixation device of claim 1, wherein the first fixator member and the second fixator member are is configured to be coupled to the fracture.

5. The fixation device of claim 4, wherein the frame is removed after the first fixator member and the second fixator member have been coupled to the fracture.

6. The fixation device of claim 1, wherein the elongate member is a first elongate member, and wherein the frame includes a second elongate member, and wherein the first carrier includes a first attachment feature configured to engage the first elongate member and a second attachment feature configured to engage the second elongate member, and wherein the first and second attachment features are not rotatable relative to the first and second elongate members.

7. The fixation device of claim 1, further comprising:
a force sensor coupled to the frame, the force sensor configured to sense forces applied to the frame by the fracture; and
a computing system configured to receive forces sensed by the force sensor and determine, based on the received sensed forces, a healing state of the fracture.

8. A method of treating a facture on a long bone having a first end and a second end, the method comprising:
positioning a first carrier along an elongate member of a frame of a fixation device such that the first carrier is oriented to be proximate the first end of the long bone when the frame is coupled is coupled to the long bone;
positioning a second carrier along the elongate member of the frame such that the second carrier is oriented to be proximate the second end of the long bone when the frame is coupled is coupled to the long bone;
coupling a first fixator member to the first carrier;
coupling a second fixator member to the second carrier; and
coupling the first fixator member and the second fixator member to the fracture.

9. The method of claim 8, wherein the fixation device is coupled to the fracture after the first and second carriers have been coupled to the elongate member and after the first and second fixator members have been coupled to the first and second carriers.

10. The method of claim 8, wherein the first carrier has a first configuration in which the first carrier is slidable along the elongate member and a second configuration in which the first carrier is fixedly coupled to the elongate member.

11. The method of claim 8, wherein the method includes adjusting a length of the elongate member using telescoping elements in the elongate member.

12. The method of claim 8, further comprising removing the frame after the first fixator member has been coupled to the fracture.

13. The method of claim 8, wherein the elongate member is a first elongate member, wherein the frame includes a second elongate member, and wherein the first carrier includes a first attachment feature configured to engage the first elongate member and a second attachment feature configured to engage the second elongate member, and wherein the first and second attachment features are not rotatable relative to the first and second elongate members.

14. The method of claim 8, further comprising manipulating bone fragments of the fracture to reduce the fracture.

15. The method of claim 14, wherein manipulating the bone fragments includes repositioning at least one of the first fixator member and the second fixator member by a longitudinal adjuster coupled to the frame.

16. The method of claim 14, wherein manipulating the bone fragments includes rotating the first fixator member relative to the first carrier.

17. The method of claim 14, wherein manipulating the bone fragments includes inserting a pin into a bone fragment of the fracture to reposition the bone fragment, the pin coupled to a third fixator member coupled to the elongate member via a third carrier.

18. A portable fixation table configured to stabilize a fracture in a long bone, the portable fixation table comprising:
a frame including an elongate member;
a first carrier configured to be coupled to a first fixator member and configured to be coupled to the elongate member; and
a second carrier configured to be coupled to a second fixator member and configured to be coupled to the elongate member;
wherein the frame is configured to be moveable between a collapsed state in which the first and second fixator members lie in a plane that is generally parallel to a plane in which the elongate member lies, and a deployed state in which the first fixator member lies in a plane that is generally perpendicular to the plane in which the elongate member lie and in which the second fixator member lies in a plane that is generally perpendicular to the plane in which the elongate member lies.
